(19) United States
(12) Patent Application Publication
(10) Pub. No.: US 2025/0075275 A1
(43) Pub. Date: Mar. 6, 2025

(54) GENE/PROTEIN EXPRESSION GUIDED MELANOMA SURGERY

(71) Applicant: The Regents of the University of California, Oakland, CA (US)

(72) Inventors: John McPherson, Oakland, CA (US); Maija Kiuru, Oakland, CA (US)

(73) Assignee: The Regents of the University of California, Oakland, CA (US)

(21) Appl. No.: 18/681,427

(22) PCT Filed: Aug. 5, 2022
(86) PCT No.: PCT/US2022/074593

§ 371 Date: Feb. 5, 2024

Related U.S. Application Data
(60) Provisional application No. 63/229,983, filed on Aug. 5, 2021.

(51) Int. Cl. C12Q 1/6886; C12Q 1/6841; G16B 25/10

(52) U.S. Cl. CPC C12Q 1/6886; G16B 25/10; C12Q 1/6841; C12Q 2600/158

(57) ABSTRACT

Provided herein are methods for locating and characterizing a melanoma tumor by detecting expression levels of one or both of S100A8 and S100A9. The provided methods are particularly useful for evaluating spatially specific properties of the tumor, and for using this information to locate the border of the tumor, surgically removing the tumor, or treat the tumor by administering a particularly determined dosage of a therapeutic agent.

### CROSS-REFERENCE TO RELATED APPLICATIONS

The present application claims priority to U.S. Provisional Application No. 63/229,983 filed Aug. 5, 2021, the full disclosure of which is incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant No. K23AR074530 awarded by the National Institute of Arthritis and Musculoskeletal and Skin Diseases of the National Institute of Health. The government has certain rights in the invention.

### BACKGROUND

Melanoma is the fifth most common cancer type in the United States and causes the vast majority of skin cancer deaths. Despite being the deadliest of the common skin cancers, melanoma is curable with early diagnosis and treatment (J. E. Gershenwald et al., CA Cancer J. Clin. 67, (2017): 472). However, histopathologic diagnosis of melanoma can be complicated by morphological mimicry, especially its early forms, by a subset of melanocytic nevi. Because development of melanoma is a stepwise process in which melanocytes accrue mutations and escape environmental controls on proliferation (J. Villanueva & M. Herlyn, Curr. Oncol. Rep. 10, (2008): 439), understanding the interaction of melanocytes with neighboring cell types is crucial to development of diagnostic tools and effective treatments. Even with recent advances in immunotherapy, however, markers predicting sustained treatment response are inadequate. Understanding the interplay between melanocytes and neighboring keratinocytes and immune cells will be crucial to the development of improved diagnostic and prognostic tools and therapeutic targets.

Many melanoma-associated genes have been identified (B. C. Bastian. Annu. Rev. Pathol. 9, (2014): 239; C. Charbel et al., J. Invest. Dermatol. 134, (2014): 1067; M. R. Roh, P. Eliades, S. Gupta. & H. Tsao, Pigment Cell Melanoma Res. 28, (2015): 661; A. H. Shain & B. C. Bastian, N. Engl. J. Med. 374, (2016): 995, A. H. Shain et al., N. Engl. J. Med. 373, (2015): 1926) and molecular tests for diagnosis and prognosis melanoma are gradually being introduced (L. E. Clarke et al., J. Cutan. Pathol. 42, (2015): 244, P. Gerami et al., J. Am. Acad. Dermatol. 72, (2015):780-5 e3; P. Gerami et al., Clin. Cancer Res. 21, (2015): 175), but markers of early melanoma development, including within the tumor microenvironment, remain lacking. In addition, although the treatment of metastatic melanoma has changed drastically since the development of immune checkpoint inhibitor therapies (D. O. Khair et al., Front. Immunol. 10, (2019): 453), biomarkers predicting durable treatment response are largely unknown. Given the heterogeneity, low cellularity, and spatial context of immune and microenvironment responses (F. Finotello & F. Eduati,. Front. Oncol. 8, (2018): 430), spatially resolved techniques are likely to outperform bulk molecular profiling for discovery of early stage and predictive biomarkers.

Furthermore, a subset of melanomas may be challenging to fully remove surgically due to ill-defined clinical borders. Most commonly such melanomas are located on functionally and/or cosmetically sensitive anatomic sites such as the acral or facial skin, where conservation of surrounding normal tissue is critical. Therefore, a more precise delineation of the tumor borders, e.g., through spatial differentiation of tumor markers, prior to surgical treatment is warranted.

Previous studies have revealed the importance of keratinocyte-derived growth factors and cell adhesion molecules in limiting melanocyte proliferation in normal skin and elucidated mechanisms by which malignant melanocytes escape this regulation (N. K. Haass, K. S. Smalley, L. Li, & M. Herlyhn, Pigment Cell Res. 18, (2005): 150; J. Villanueva & M. Herlyn, Curr. Oncol. Rep. 10, (2008): 439). However, these experiments relied on the use of co-culture systems or heterologous expression of keratinocyte-derived genes in melanocytes, neither of which capture the spatial element of melanocyte-keratinocyte interactions in situ. Furthermore, single-cell RNA sequencing (scRNAseq) studies on melanoma have largely focused on melanoma metastases, overlooking the keratinocyte microenvironment of primary melanomas (L. Jerby-Amon et al., Cell 175, (2018): 984; I. Tirosh et al., Science 352, (2016): 189). Since scRNAseq rely on fresh tissue, studies on benign melanocytic tumors in humans are also lacking.

In view of these and other challenges associated with methods using many existing melanoma biomarkers for tumor identification, diagnosis, and treatment, there is a need in the art for improved techniques based on markers with, e.g., a high degree of spatial discrimination. The present disclosure addresses this need and provides associated and other advantages.

### BRIEF SUMMARY

In general, provided herein are methods for locating and treating melanoma tumors, in part by assessing the expression levels of particular marker genes associated with the tumors. For example, methods disclosed herein can utilize S100A8/S100A9 expression within the epidermis associated with melanoma to more precisely map melanoma tumor borders and determine a radius of excision to guide a surgical treatment. The S100A8 and S100A9 genes and gene products are shown to be expressed in epidermal keratinocytes associated with melanoma but not with benign tumors. As epidermal keratinocytes form the uppermost layer of the skin, this differential behavior allows a non-invasive approach for the detection of S100A8/S100A9 expression and hence the underlying tumor. The detection of S100A8/S100A9 expression can thus be used, e.g., to map the tumor borders and determine the radius/size of the surgical margin, or to determine a therapeutically effective dosage amount of an agent targeting the tumor.

In one aspect, the disclosure provides a method of mapping a border of a melanoma tumor in a subject. The method includes applying an adhesive patch to a skin area of the subject, where the skin area includes at least a portion of a skin surface overlying the melanoma tumor, and where a portion of the skin area does not include the skin surface overlying the melanoma tumor. The method further includes removing the adhesive patch from the subject. The method further includes detecting an expression level of one or both of S100A8 and S100A9 in each of a plurality of regions of a sample of the skin area adhered to the adhesive patch. The method further includes locating at least a portion of the border of the melanoma tumor by comparing the expression levels in each of the plurality of sample regions.

In another aspect, the disclosure provides a method for the surgical removal of a melanoma tumor from a subject. The method includes applying an adhesive patch to a skin area of the subject, where the skin area includes at least a portion of a skin surface overlying the melanoma tumor, and where a portion of the skin area does not include the skin surface overlying the melanoma tumor. The method further includes removing the adhesive patch from the subject. The method further includes detecting an expression level of one or both of S100A8 and S100A9 in each of a plurality of regions of a sample of the skin area adhered to the adhesive patch. The method further includes locating at least a portion of the border of the melanoma tumor by comparing the expression levels in each of the plurality of sample regions. The method further includes determining, based on the located portion of the border of the melanoma tumor, an outline of a tissue mass to be excised from the subject, where the tissue mass includes at least a portion of the melanoma tumor. The method further includes excising the tissue mass from the subject.

In another aspect, the disclosure provides a method for the treatment of a melanoma tumor in a subject. The method includes applying an adhesive patch to a skin area of the subject, where the skin area includes at least a portion of an external surface of the melanoma tumor, and where a portion of the skin area does not include an external surface of the melanoma tumor. The method further includes removing the adhesive patch from the subject. The method further includes detecting an expression level of one or both of S100A8 and S100A9 in each of a plurality of regions of a sample of the skin area adhered to the adhesive patch. The method further includes determining, based on the expression level in at least one of the plurality of regions, a dosage amount of a therapeutic agent, where the dosage amount is effective for the treatment of the melanoma tumor. The method further includes administering to the subject the dosage amount of the therapeutic agent.

In another aspect, the disclosure provides a method for the treatment of a suspected melanoma tumor in a subject. The method includes applying an adhesive patch to a skin area of the subject, where the skin area includes at least a portion of an external surface of the suspected melanoma tumor, wherein the suspected melanoma tumor is not grossly visible, and where a portion of the skin area does not include an external surface of the melanoma tumor. The method further includes removing the adhesive patch from the subject. The method further includes detecting an expression level of one or both of S100A8 and S100A9 in each of a plurality of regions of a sample of the skin area adhered to the adhesive patch. The method further includes determining, based on the expression level in at least one of the plurality of regions, a dosage amount of a therapeutic agent, where the dosage amount is effective for the treatment of the suspected melanoma tumor. The method further includes administering to the subject the dosage amount of the therapeutic agent.

### BRIEF DESCRIPTION OF THE DRAWINGS

FIG. 1 is a schematic illustration of comparisons enabled by the experimental design for spatially resolving mRNA biomarkers in formalin-fixed paraffin-embedded (FFPE) samples from four pathologically defined tumor types.

FIG. 2 is an illustration of the pathway content of the 1,412-target (4,998-probe) gene panel for digital spatial profiling (DSP) with next-generation sequencing (NGS) readout. The panel content is approximately 35% immune-related, 40% tumor-related, and 20% microenvironment-related, with 1% housekeeping genes and 3% negative probes.

FIG. 3 is a schematic illustration of probe design for DSP with NGS readout. Each probe contains an antisense sequence that hybridizes to target mRNA, a photocleavable linker (circle), an RNA ID that identifies the mRNA target, and a unique molecular identifier (UMI) to allow removal of PCR duplicates when converting reads to digital counts. DSP probe pools target each gene with 1-10 probes that hybridize to different sequences along the mRNA transcript and contain >80 negative probes that target scrambled or non-human sequences.

FIG. 4 is an illustration of the experimental workflow for DSP with NGS readout. Collected oligos are PCR-amplified using indexing primers to preserve region-of-interest (ROI) identity, pooled, purified, and sequenced.

FIG. 5 is an illustration of an exemplary ROI selection process. Top images: ROIs selected by a pathologist based on enrichment for melanocytes, keratinocytes, or immune cells in hematoxylin- and eosin-(H&E) stained section. Bottom images: ROIs collected from a serial section during DSP. Fluorescent antibodies to melanocyte markers S100B and PMEL, T cell marker CD3, lymphocyte marker CD45, and DNA stain SYTO 13 were used as visualization markers during DSP to guide matching of ROIs to the H&E sections.

FIG. 6 presents H&E and matching DSP images of example ROIs for each of six categories defined in a study of the influence of cell type and tumor type on the expression profile of each ROI. All ROIs selected were 200-μm circles.

FIG. 7 is a ternary plot displaying cell type composition (% melanocyte, keratinocyte, or immune) of each ROI as determined by pathologist evaluation. Shaded regions indicate assignment to the ROI categories shown in FIG. 6.

FIG. 8 presents a series of boxplots of upper quartile-normalized counts by tumor type for selected melanomagenesis-associated genes known to be enriched in melanocytes (PRAME and PMEL), keratinocytes (KRT14 and CXCL14) or immune infiltrates (PTPRC and CXCL9).

FIG. 9 is a correlation matrix showing pairwise correlation coefficients (Pearson R) between all ROIs using scaled normalized counts for the 923 genes detected in the experiment of FIGS. 6-8 (489 genes below the detection threshold in all 134 ROIs were removed prior to clustering). The five largest clusters (helust method) are boxed and named according to their predominant cell type (M1-2 for melanocyte-rich, 11-2 for immune-rich. K1 for keratinocyte-rich).

FIG. 10 is a volcano plot comparing gene expression in M1 ROIs to all other ROIs classified as Keratinocyte-rich, Melanocyte-rich, Keratinocyte/melanocyte, or Mixed. Significance (−log 10 of p value) was determined by linear regression with a term for random effects from inter-tissue variation. Genes were only considered if they were above the detection threshold in at least three ROIs and gene names are only shown if the gene was below the detection threshold in all common nevus ROIs.

FIG. 11 is a volcano plot comparing gene expression in 11 ROIs to all other ROIs classified as Immune or Immune/melanocyte. Significance (−log 10 of p value) was determined by linear regression with a term for random effects from inter-tissue variation. Genes were only considered if they were above the detection threshold in at least three ROIs and gene names are only shown if the gene was below the detection threshold in all common nevus ROIs.

FIG. 12 is a graph of a UMAP analysis comparing the spatial expression profiles of all 923 genes detected in at least one ROI. Highlighted dots were enriched in the indicated ROI type as determined by linear regression (FDR <0.01). Top hits from the volcano plots in FIGS. 10 and 11 are indicated.

FIG. 13 is a graph of a UMAP analysis comparing the spatial expression profiles of all 923 genes detected in at least one ROI. Highlighted dots were enriched in the indicated tumor type as determined by linear regression (FDR <0.01). Selected genes adjacent to top hits from the from the FIGS. 10 and 11 volcano plots are indicated in UMAP space.

FIG. 14 is a ridgeline plot of normalized counts for selected genes in all ROIs, organized by tumor type (CN=common nevus, DN=dysplastic nevus, MIS=melanoma in situ, MM=melanoma). Line indicates the detection threshold in each ROT.

FIG. 15 is a ridgeline plot of normalized counts for selected genes in all ROIs, organized by tumor type (CN=common nevus, DN=dysplastic nevus, MIS=melanoma in situ, MM=melanoma). Line indicates the detection threshold in each ROI.

FIG. 16 is a volcano plot comparing gene expression in the subset of ROIs classified as melanoma in situ by a pathologist vs. all other ROIs in K1/M2. Significance (−log 10 of p value) determined by linear regression with a term for random effects from inter-tissue variation. Genes were only considered if they were above the detection threshold in at least three ROIs, and gene names are only shown if the gene was not detected in any common nevus ROIs.

FIG. 17 presents a representative immunohistochemistry (IHC) image (left panel) and corresponding H&E image (right panel) showing that S100A8 is expressed by keratinocytes (arrowhead) rather than melanocytes (arrow).

FIG. 18 is a ternary plot of S100A8 expression in all ROIs, with a zoomed-in view of Keratinocyte/melanocyte ROIs at left.

FIG. 19 presents H&E images of selected ROIs containing a mixture of keratinocytes and melanocytes, and a graph of normalized S100A8 counts plotted against keratinocyte score and indicated by tumor type.

FIG. 20 is a representative image of S100A8 IHC of invasive melanoma, showing that S100A8 is expressed by keratinocytes in invasive melanoma. Letter “e” indicates epidermal layer containing keratinocytes and letter “t” indicates tumor.

FIG. 21 is a representative image of S100A8 H&E staining corresponding to the IHC image of FIG. 20, showing that S100A8 is expressed by keratinocytes in invasive melanoma. Letter “e” indicates epidermal layer containing keratinocytes and letter “t” indicates tumor.

FIG. 22 is a representative image of S100A8 IHC of melanoma in situ showing that S100A8 is expressed by keratinocytes in melanoma in situ. Letter “e” indicates epidermal layer containing keratinocytes and letter “t” indicates tumor.

FIG. 23 is a representative image of S100A8 H&E staining corresponding to the IHC image of FIG. 22, showing that S100A8 is expressed by keratinocytes in melanoma in situ. Letter “e” indicates epidermal layer containing keratinocytes and letter “t” indicates tumor.

FIG. 24 is a representative image of S100A8 IHC of dysplastic nevus showing that S100A8 is not expressed by keratinocytes in dysplastic nevus. Letter “e” indicates epidermal layer containing keratinocytes and letter “t” indicates tumor. Melanin pigment is present in dysplastic nevus.

FIG. 25 is a representative image of S100A8 H&E staining corresponding to the IHC image of FIG. 22, showing that S100A8 is not expressed by keratinocytes in dysplastic nevus. Letter “e” indicates epidermal layer containing keratinocytes and letter “t” indicates tumor.

FIG. 26 is a representative image of S100A8 IHC of common nevus showing that S100A8 is not expressed by keratinocytes in common nevus. Letter “e” indicates epidermal layer containing keratinocytes and letter “t” indicates tumor.

FIG. 27 is a representative image of S100A8 H&E staining corresponding to the IHC image of FIG. 22, showing that S100A8 is not expressed by keratinocytes in common nevus. Letter “e” indicates epidermal layer containing keratinocytes and letter “t” indicates tumor.

FIG. 28 is a graph of an S100A8 receiver operating characteristic (ROC) curve for in situ or invasive melanoma. AUC, area under the ROC curve.

FIG. 29 is a low-power image of S100A8 IHC of melanoma with in situ and invasive components and an area of uninvolved skin (upper panel). S100A8 is expressed by keratinocytes in the epidermis directly associated with melanoma (insets 1 and 3), but not in the uninvolved epidermis (inset 2). Letter “e” indicates epidermal layer containing keratinocytes and letter “t” indicates tumor. Melanin pigment is present in association with invasive melanoma (*).

FIG. 30 is a ridgeline plot of normalized counts for selected genes in all ROIs of the experiment of FIGS. 16-19, organized by tumor type.

FIG. 31 is a graph showing that gene signatures depicted in FIG. 14 are predictive of cell type and case type. Principal components analysis (PCA) was performed on log 2-transformed upper quartile-normalized counts using the gene signatures of FIG. 14. Principal component 1 (PC1) differentiates melanocyte-containing ROIs in melanoma cases from all other ROIs.

FIG. 32 is a graph showing that gene signatures depicted in FIG. 15 are predictive of cell type and case type. PCA was performed on log 2-transformed upper quartile-normalized counts using the gene signatures of FIG. 15. PC1 differentiates immune cell-rich ROIs from melanoma and melanoma in situ cases from all other ROIs.

FIG. 33 is a schematic illustration of the semi-quantitative scoring of S100A8 expression. S100A8 staining was scored as shown based on the percentage of epidermis stained associated with the tumor: 1=0 to 5%; 2=6% to 25%: 3=26% to 50%; 4=51% to 75%; 5=>75%,

FIG. 34 presents a series of images representative of S100A8 staining scores 1-5. (a-d) Score 1: Representative images of S100A8 IHC staining score 1 (c,d) and corresponding H&E staining (a,b) in a common nevus. (e-h) Score 2; Representative images of S100A8 IHC staining score 2 (g,h) and corresponding H&E staining (e,f) in a dysplastic nevus. (i-j) Score 3: Representative images of S100A8 IHC staining score 3 (k,l) and corresponding H&E staining (i,j) in melanoma in situ; An associated melanocytic nevus is present. (m-p) Score 4; Representative images of S100A8 IHC staining score 4 (o,p) and corresponding H&E staining (m,n) in melanoma in situ. (q-t) Score 5; Representative images of S100A8 IHC staining score 5 (s,t) and corresponding H&E staining (q,r) in invasive melanoma. Tumors were double-stained for S100A8 and PRAME. If present, S100A8 is expressed by keratinocytes and scattered immune cells and PRAME by melanocytes. Magnification 40× (a,c.e,g,i,k,m,o,q,s) or 100× (b,d,f,hj,l,n,p,r,t).

FIG. 35 presents a series of images showing that S100A9 is expressed in epidermis directly associated with melanoma, but not in uninvolved areas of epidermis. (a-d) Representative images of S100A9 IHC (left panel) and corresponding H&E staining (right panel) of invasive melanoma in (a) and (b) and common nevus in (c) and (d). S100A9 is expressed by keratinocytes in invasive melanoma in (a), but not in common nevus in (c).

### DETAILED DESCRIPTION

Provided herein are methods involving locating and/or treating a melanoma tumor in the body of a subject. The methods make use of the observation that epidermal keratinocytes associated with melanoma express S100A8 and/or S100A9. This behavior has been demonstrated with spatial RNA expression profiling and immunohistochemical protein expression analysis of numerous melanocytic tumors. In particular, S100A8 is prominently expressed by keratinocytes within the tumor microenvironment during melanoma growth, but not in benign tumors. This expression pattern has been confirmed to also be true for S100A8's binding partner, S100A9, suggesting that injury to the epidermis may be an early and readily detectable indicator of melanoma development. Together, these findings establish a framework for high-plex, spatial, and cell type-specific resolution of gene expression applicable to characterization of melanoma tumors, the early diagnosis of which is critical for improved survival.

The damage-associated molecular pattern (DAMP) S100A8 is a known melanoma marker having multiple roles in promoting immune responses and inflammation (T. Nukui et al., J. Cell Biochem. 104, (2008): 453; G. Srikrishna, J. Innate Immun. 4, (2012): 31). It is most well-known as part of the S100A8/A9 complex (calprotectin), which is canonically expressed and secreted by neutrophils, monocytes and macrophages (C. Gebhardt, J. Nemeth, P. Angel, & J. Hess, Biochem. Pharmacol. 72, (2006): 1622). S100A81A9 is also upregulated in a number of inflammatory disorders such as psoriasis and cystic fibrosis (C. Gebhardt, J. Nemeth, P. Angel, & J. Hess, Biochem. Pharmacol. 72, (2006): 1622; T. Nukui et al., J. Cell Biochem. 104, (2008): 453) and expressed in a variety of epithelial tumor types (C. Gebhardt, J. Nemeth, P. Angel, & J. Hess, Biochem. Pharmacol. 72, (2006): 1622), where it is associated with invasiveness and poor prognosis (K. Arai et al., Curr. Cancer Drug Targets 8, (2008): 243). While upregulation of S100A8A9 in melanoma has been established using bulk methods (L. E. Clarke et al., J. Cutan. Pathol. 42, (2015): 244; M. Kunz et al., Oncogene 37, (2018): 6136; N. B. Wagner et al., J. Immunother. Cancer 7, (2019): 343), previous studies have assumed that it is expressed by immune cells (L. E. Clarke et al., J. Cutan. Pathol. 42, (2015): 244; N. B. Wagner et al., J. Immunother. Cancer 7, (2019): 343).

The analysis disclosed herein demonstrates instead a keratinocyte-derived origin of S100A8/A9 during melanoma development. This observation can provide an explanation for why S100A8/A9 is strongly detected in primary melanomas but not metastases (T. F. Xiong, F. Q. Pan, & D. Li, Melanoma Res. 29, (2019): 23). Furthermore, it emphasizes the importance of understanding a biomarker's cellular origin, as non-cutaneous metastasis and micro-dissected tumors may lack keratinocytes and thus produce falsely low expression levels of S100A8-A9 in current commercially available tests. In addition, the results suggest a potential role for S100A8 immunohistochemistry (IHC) as an ancillary test for the diagnosis of melanoma, especially as IHC-based testing can be readily adopted for use in most pathology laboratories. Furthermore, expression of S100A8-A9 specifically at the skin surface in early melanoma can be exploited to increase the sensitivity of an adhesive patch biopsy assay, such as that available for PRAME (P. Gerami et al., J. Am. Acad. Dermatol. 76, (2017): 114).

Such biopsy assays can have high significance for treatment methods, because melanoma tumor borders are typically defined clinically prior to surgical removal. As many melanoma tumors have clinically ill-defined borders and microscopic extension, initial surgical margin may not be adequate to fully remove the tumor, particularly in areas that are functionally and/or cosmetically sensitive. Among the advantages of the methods provide herein is that tumor borders can be more accurately defined using non-invasive analysis of S100A8/S100A9 expression, resulting in improved surgical clearance while preserving normal tissue surrounding the tumor. Ultimately, this can result in reduced tumor recurrence and reduced morbidity and mortality in melanoma.

For example, the non-invasive detection of tumor borders can involve applying an adhesive patch on the tumor and surrounding skin. To preserve spatial information for mapping, the adhesive patch can be analyzed segmentally for RNA expression or directly on the adhesive patch with immunohistochemistry. The adhesive patch can also be applied in such a way that it leaves registration marks on the skin. The test results, i.e., the expression landscape, as determined from the adhesive patch can thus be registered to the skin surface using the registration marks. This can allow determination of tumor borders that can be used to define radius or size of the surgical margin prior to tumor removal. In other embodiments, the tumor characterization by specific expression profiling can be used to guide treatment of the melanoma by administration of a therapeutic agent, rather than or in addition to treatment by surgical excision. The therapeutic agent administration protocol can be defined in direct response to the results of the expression profiling. For example, the dosage of the therapeutic agent can be determined based on the level of expression associated with the tumor.

### I. Methods of Mapping Melanoma Tumor Borders

In one aspect, a method of mapping a border of a melanoma tumor in a subject is provided. The method disclosed herein provides surprising improvements in detecting the tumor border, e.g., the border between the subject's skin area overlying the melanoma tumor, and the subject's skin area surrounding, rather than overlying, the tumor. These borders can be difficult to ascertain with other clinical methods, such as those using only visual inspection or imaging with various modalities. Advantageously, the provided method, based instead on different S100A8 and S100A9 expression levels between tumor overlying areas of the skin and the surrounding areas of the skin, can detect such borders with a higher degree of specificity and accuracy.

The subject having the melanoma tumor in all methods disclosed herein is generally a vertebrate, preferably a mammal, and more preferably a human. Mammals that are suitable subjects of the methods include, but are not limited to, murines, simians, humans, farm animals, sport animals, and pets. In some embodiments, the subject is a human medical patient.

The provided methods generally involve sampling a skin area of the subject, where the skin area includes at least some skin surface associated with, e.g., overlying, the melanoma tumor, and at least some other skin surface adjacent to the skin surface that is associated with or overlying the tumor. In this way, the sampled skin area includes at least some border between a tumor overlying region of the skin and a surrounding region of the skin. The skin area that is sampled by the method need not, but in some embodiments can, include all of the subject's skin surface that is overlying the tumor. Also, the fraction of the sampled skin area that does not include skin surface overlying the tumor can vary, as long as some amount of the surrounding skin region is included in the sampled skin area so that the sample can be used to locate some portion of the tumor border.

The skin area can include a percentage of the subject's skin surface overlying the melanoma tumor that is, for example, between 0 and 60%, between 10% and 70%, between 20% and 80%, between 30% and 90%, or between 40% and 100%. In terms of lower limits, the skin area can include a percentage of the skin surface overlying the melanoma tumor that is greater than 10%, greater than 20%, greater than 30% greater than 40%, greater than 50%, greater than 60%, greater than 70%, greater than 80%, or greater than 90%. In terms of lower limits, the skin area can include a percentage of the skin surface overlying the melanoma tumor that is less than 90%, less than 80%, less than 70%, less than 60%, less than 50%, less than 40%, less than 30%, less than 20%, or less than 10%. In some embodiments, the skin area includes 100% of the subject's skin surface that is overlying the melanoma tumor.

The percentage of the skin area that does not include any of the skin surface overlying the melanoma tumor can be, for example, between 0 and 60%, between 10% and 70%, between 20% and 80%, between 30% and 90%, or between 40% and 100%. In terms of lower limits, the percentage of the skin area not including any of the skin surface overlying the melanoma tumor can be greater than 10%, greater than 20%, greater than 30% greater than 40%, greater than 50%, greater than 60%, greater than 70%, greater than 80%, or greater than 90%. In terms of lower limits, the percentage of the skin area not including any of the skin surface overlying the melanoma tumor can be less than 90%, less than 80%, less than 70%, less than 60%, less than 50%, less than 40%, less than 30%, less than 20%, or less than 10%.

In some embodiments, the skin area of the subject is sampled by applying an adhesive patch to the skin area. The adhesive patch is generally configured such that the skin area, or at least a substantial portion thereof, adheres or otherwise connects to the adhesive patch. The adhering or connection of the skin area to the adhesive patch is strong enough that at least some adhered or connected skin remains with the adhesive patch when the adhesive patch is removed from the subject. In this way, the adhesive patch retains a sample of the skin area upon removal of the patch from the subject. This sample can then be subjected to further analysis, e.g., to detect S100A8 and/or S100A9 expression levels therein. In some embodiments, the sample retained by the adhesive patch is analyzed while attached or connected to the adhesive patch. In some embodiments, the sample retained by the adhesive is removed from the adhesive patch prior to analysis.

In alternative embodiments, the skin area of the subject is sampled using other techniques. The skin area can be sampled through other means used to remove cells from the skin area for ex vivo analysis. For example, cells can be scraped from the skin area for further analysis. The skin area can also be sampled by analyzing the skin area of the subject in vivo. For example, the skin area can be sampled by applying to the skin area one or more detection compounds linked to imaging agents for detecting an expression level of one or both of S100A8 and S100A9. Suitable detection agents can include, for example, probes with labeled complementary polynucleotides, e.g. probes that target mRNA transcripts specific to the S100A8 gene or the S100A9 gene. Suitable detection agents can additionally or alternatively include S100A8 monoclonal antibodies and/or S100A9 monoclonal antibodies. The imaging agents can include, for example, fluorescent markers. The applying of the detection compounds can be accomplished via injection, a transdermal patch, or other means recognized by those of skill in the art for delivering material, e.g., detection compounds and/or imaging agents, to the skin area.

For embodiments involving applying an adhesive patch to the skin area of the subject, the adhesive patch can be removed from the subject after a residence time on the skin area selected to be adequate for retaining a sample of the skin area with the adhesive patch as described above. Suitable residence times can depend on the physical structure and/or chemical composition of the surface of the adhesive patch applied to the subject's skin area. The adhesive patch can be removed from the subject after a residence time that is, for example, between 1 second and 60 minutes, e.g., between 1 second and 2 minutes, between 2 seconds and 5 minutes, between 5 seconds and 12 minutes, between 12 seconds and 26 minutes, or between 26 seconds and 60 minutes. In terms of upper limits, the adhesive patch can be removed from the subject after a residence time that is less than 60 minutes, e.g., less than 26 minutes, less than 12 minutes, less than 5 minutes, less than 2 minutes, less than 1 minute, less than 26 seconds, less than 12 seconds, less than 5 seconds, or less than 2 seconds. In terms of lower limits, the adhesive patch can be removed from the subject after a residence time that is greater than 1 second, e.g., greater than 2 seconds, greater than 5 seconds, greater than 12 seconds, greater than 26 seconds, greater than 1 minute, greater than 2 minutes, greater than 5 minutes, greater than 12 minutes, or greater than 26 minutes. Longer residence times, e.g., greater than 60 minutes, and shorter residence times, e.g., less than 1 second, are also contemplated.

The sample of subject's skin area, whether the sample is removed from the subject or instead remains on the body of the subject, is then analyzed in a region-by-region fashion. The sample is divided into a plurality of regions for the analysis. In some embodiments, the division of the sample into the plurality of regions is a physical division, in which different regions are removed from the overall sample. In some embodiments, the division of the sample is a nominal division, in which different regions are defined but remain with the sample.

The number, shape, and location of the plurality of regions are selected to provide adequate coverage of the sample for identifying at least a portion of the border of the melanoma tumor. The sample can be divided into a number of regions that is, for example, between 5 and 500, e.g., between 5 and 79, between 8 and 130, between 13 and 200, between 20 and 320, or between 32 and 500. In terms of upper limits, the sample can be divided into a number of regions that is less than 500. e.g., less than 320, less than 200, less than 130, less than 79, less than 50, less than 32, less than 20, less than 13, or less than 8. In terms of lower limits, the sample can be divided into a number of regions that is greater than 5, e.g., greater than 8, greater than 13, greater than 20, greater than 32, greater than 50, greater than 79, greater than 130, greater than 200, or greater than 320. Larger numbers of regions, e.g., greater than 500, and smaller numbers of regions, e.g., less than 5, are also contemplated.

Each of the plurality of regions can have a shape that is substantially square or rectangular. Each of the plurality of regions can have a shape that is substantially circular or oval. The plurality of regions can be arranged in a regular pattern, e.g., a grid. The plurality of regions can be arranged irregularly. In some embodiments, the plurality of regions share an identical size. In some embodiments, at least one of the plurality of regions has a size different from that of another of the plurality of regions. In some embodiments, the plurality of regions share an identical shape. In some embodiments, at least one of the plurality of regions has a shape different from that of another of the plurality of regions.

In some embodiments, the combined area of the plurality of regions includes all of the sample of subject's skin area, such that each location on the sample is within one of the plurality of regions. In some embodiments, the combined area of the plurality of regions does not include all of the sample of subject's skin area, such that at least one location on the sample is outside each of the plurality of regions. The percentage of the sample included in the plurality of regions can be, for example, between 0 and 60%, between 10% and 70%, between 20% and 80%, between 30% and 90%, or between 40% and 100%. In terms of lower limits, the percentage of the sample included in the plurality of regions can be greater than 10%, greater than 20%, greater than 30% greater than 40%, greater than 50%, greater than 60%, greater than 70%, greater than 80%, or greater than 90%. In terms of lower limits, the percentage of the sample included in the plurality of regions can be less than 90%, less than 80%, less than 70%, less than 60%, less than 50%, less than 40%, less than 30%, less than 20%, or less than 10%.

For embodiments in which the sample of subject's skin area is removed from the subject for ex vivo analysis, subject skin area regions corresponding to the plurality of sample regions are identified. In this way, the analytical result measured for each of the plurality of regions can provide information about a related skin area region for the subject. In some embodiments, the identification of subject skin area regions corresponding to the sample regions is facilitated by registration marks on the adhesive patch applied to the subject skin area. The registration marks can be configured to transfer indicia from the adhesive patch to the subject skin area when the adhesive patch is applied to the skin area. Accordingly, when the adhesive patch is removed from the skin area, indicia remain on the skin area reflecting the location and configuration of the adhesive patch registration marks. A correlation of the transferred indicia to the registration marks can then be used to identify subject skin area regions corresponding to the plurality of sample regions.

The expression level of one or both of S100A8 and S100A9 is detected in each of a plurality of sample regions. In some embodiments, the S100A8 expression level is measured, and the S100A9 expression level is not measured. In some embodiments, the S100A9 expression level is measured and the S100A8 expression level is not measured. In some embodiments, the S100A8 expression level and the S100A9 expression level are measured. In some embodiments, the expression level is detected by measuring RNA concentration, e.g., the concentration of mRNA corresponding the genetic sequence of the S100A8 gene or the S100A9 gene. The detection of the expression level can include the use of probes with labeled complementary polynucleotides, e.g. probes that target mRNA transcripts specific to the S100A8 gene or the S100A9 gene. In some embodiments, the expression level is detected by measuring protein concentration, e.g., the concentration of the S100A8 protein or the S100A9 protein. The detection of the expression level can include the use of antibodies having binding affinity for epitopes for the S100A8 protein or the S100A9 protein. The detection can include the use of S100A8 monoclonal antibodies and/or S100A9 monoclonal antibodies. The antibodies can be linked to imaging agents. e.g., the antibodies can be conjugated to a fluorescence marker. In some embodiments, the detection includes the use of labeled, e.g., fluorescently labeled, secondary antibodies having binding affinity for primary S100A8 antibodies and/or primary S100A9 antibodies. Other detection techniques well-understood in the art are also contemplated.

The expression levels detected in the plurality of regions of the sample are compared to one another and/or to a threshold value, allowing for the locating of at least a portion of the border of the melanoma tumor. In some embodiments, the comparing of the expression levels involves classifying each region of the sample skin area as being either a tumor overlying region or a surrounding region. In some embodiments, the classification of a skin area region is based on a comparison between a threshold expression level and the expression level in the sample region corresponding to the skin area region. For example, if the expression level in a sample region is greater than a threshold expression level, then the subject skin area region corresponding to the sample region is classified as being a tumor overlying region. Similarly, if the expression level in the sample region is less than the threshold expression level then the subject skin area region corresponding to the sample region is classified as being a surrounding region. In some embodiment, the threshold expression level has a predetermined value, e.g., a value based on historical measurements associated with other subjects and/or tumors. In some embodiments, the threshold expression level has a value calculated using a mathematical function of the expression levels detected in the plurality of sample regions.

In some embodiments, the classification of skin area regions is based on a comparison between a threshold expression level and the difference in expression levels in sample regions corresponding to the skin area regions. For example, if the expression level in a first sample region exceeds the expression level in a second sample region by more than a threshold amount, then a first sample skin area corresponding to the first sample region is classified as being a tumor overlying region, and a second sample skin area corresponding to the second sample region is classified as being a surrounding region. Alternatively, if the expression level in a first sample region exceeds the expression level in a second sample region by more than a threshold percentage, then a first sample skin area corresponding to the first sample region is classified as being a tumor overlying region, and a second sample skin area corresponding to the second sample region is classified as being a surrounding region. In some embodiment, the threshold amount or percentage has a predetermined value, e.g., a value based on historical measurements associated with other subjects and/or tumors. In some embodiments, the threshold amount or percentage has a value calculated using a mathematical function of the expression levels detected in the plurality of sample regions.

The classification of subject skin area regions as being either tumor overlying regions or surrounding regions can allow at least a portion of the border of the melanoma tumor to be located. In some embodiments, the border, or a portion thereof, is located in an area between one or more tumor overlying regions and one or more surrounding regions. In some embodiments, the border, or a portion thereof, is located at one or more common edges between a tumor overlying region and a surrounding region.

In some embodiments, and as described in further detail above, the skin area of the subject that is sampled does not include all of the skin overlying the melanoma tumor of the subject. Accordingly, in such embodiments the sample cannot be used to locate the entire border of the tumor. In part to address this issue, in some embodiments two or more skin areas of the subject are sampled to locate different portions of the tumor border. The skin areas can therefore be different from one another to cover different portions of tumor overlying skin and tumor surrounding skin. Alternatively, the skin areas associated with two or more samples can be identical or substantially identical to one another, and the results from each sample can be used to independently confirm other sample results.

### II. Methods of Surgically Removing a Melanoma Tumor Borders

In another aspect, a method of surgically removing a melanoma tumor from a subject is provided. The method disclosed herein provides surprising improvements in the surgical removal of a melanoma tumor because the border of the tumor can be located with greater accuracy and precision. By removing a section of tissue having a sufficiently large outer edge, i.e., margin, composed of normal non-cancerous cells, a surgical operation has an increased chance of removing all of the tumor from the subject. Conversely, by removing a section of tissue having a sufficiently small margin, a surgical operation has a decreased chance of negatively impacting sensitive anatomical sites of the subject.

Advantageously, the provided method can allow a surgeon to determine, based on one or more located portions of the border of the melanoma tumor, an outline of a tissue mass to be excised from the subject. This determined outline can then be used by the surgeon to excise a tissue mass from the subject, where the tissue mass includes at least a portion of the melanoma tumor, and can also include a margin of sufficient and beneficial dimensions. The portion of the melanoma tumor included in the excised tissue mass can be, for example, between 0 and 60%, between 10% and 70%, between 20% and 80%, between 30% and 90%, or between 40% and 100%. In terms of lower limits, the percentage of the tumor in the excised tissue can be greater than 10%, greater than 20%, greater than 30% greater than 40%, greater than 50%, greater than 60%, greater than 70%, greater than 80%, or greater than 90%. In terms of lower limits, the percentage of the tumor in the excised tissue can be less than 90%, less than 80%, less than 70%, less than 60%, less than 50%, less than 40%, less than 30%, less than 20%, or less than 10%.

### III. Methods of Treating a Melanoma Tumor

In another aspect, a method of treating a melanoma tumor in a subject is provided. The method disclosed herein provides surprising improvements in the treatment of melanoma tumors because the expression levels detected in the plurality of regions sampled as described in further detail above can accurately and precisely characterize the disease status of the tumor as whole and of different sub-regions of the tumor. Advantageously, the provided method is also applicable to characterizing and treating not only melanoma tumors that are readily apparent through, e.g. visible examination, but also suspected tumors not grossly visible.

In some embodiments, the expression level detected in at least one of the plurality of sample regions is used to determine a dosage amount of a therapeutic agent to be administered to the subject. In some embodiments, the expression levels detected in all of the plurality of sample regions is used to determine a dosage amount of a therapeutic agent to be administered to the subject. The dosage amount is one determined, based on the one or more detected expression levels, to be effective in treating the tumor. In some embodiments, the expression level detected in two or more of the plurality of sample regions is used to determine a location for administration of a therapeutic agent to a subject. For example, a therapeutic agent can be administered specifically to one or more subject skin areas classified as tumor overlying regions.

### IV. Embodiments

The following embodiments are contemplated. All combinations of features and embodiments are contemplated.

Embodiment 1: A method of mapping a border of a melanoma tumor in a subject, the method comprising: applying an adhesive patch to a skin area of the subject, wherein the skin area comprises at least a portion of a skin surface overlying the melanoma tumor, and wherein a portion of the skin area does not comprise the skin surface overlying the melanoma tumor; removing the adhesive patch from the subject; detecting an expression level of one or both of S100A8 and S100A9 in each of a plurality of regions of a sample of the skin area adhered to the adhesive patch and locating at least a portion of the border of the melanoma tumor by comparing the expression levels in each of the plurality of sample regions.

Embodiment 2: An embodiment of embodiment 1, wherein the detecting of the expression level comprises measuring a concentration of one or both of an S100A8 RNA sequence and an S100A9 RNA sequence.

Embodiment 3: An embodiment of embodiment 2, wherein the measuring comprises probing the sample region with polynucleotides each independently linked to an imaging agent and having a sequence substantially complementary to that of the S100A8 RNA sequence or the S100A9 RNA sequence.

Embodiment 4: An embodiment of embodiment 1, wherein the detecting of the expression level comprises measuring a concentration of one or both of S100A8 protein and S100A9 protein.

Embodiment 5: An embodiment of embodiment 4, wherein the detecting of the expression level comprises probing the sample region with antibodies each independently linked to an imaging agent and having binding affinity to the S100A8 protein or the S100A9 protein.

Embodiment 6: An embodiment of any of the embodiments of embodiment 1-5, wherein the locating comprises: identifying regions of the skin area, each independently corresponding to one of the plurality of regions of the sample.

Embodiment 7: An embodiment of embodiment 6, wherein the adhesive patch comprises registration marks, wherein the applying of the adhesive patch comprises transferring indicia of registration marks from the adhesive patch to the skin area, and wherein the identifying comprises: correlating the transferred indicia to the registration marks.

Embodiment 8: An embodiment of embodiment 6 or 7, wherein the identifying comprises: classifying a first region of the skin area as a tumor overlying region, wherein the detected expression level in the region of the sample corresponding to the first region of the skin area is greater than a first predetermined threshold; and classifying a second region of the skin area as a surrounding region, wherein the detected expression level in the region of the sample corresponding to the second region of the skin area is less than a second predetermined threshold.

Embodiment 9: An embodiment of embodiment 6 or 7, wherein the identifying comprises: classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold amount.

Embodiment 10: An embodiment of embodiment 6 or 7, wherein the identifying comprises: classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold percentage.

Embodiment 11: An embodiment of any of the embodiments of embodiment 8-10, wherein the first region of the skin area and the second region of the skin area share a common edge, and wherein the at least a portion of the border of the melanoma tumor comprises the common edge.

Embodiment 12: An embodiment of any of the embodiments of embodiment 1-11, wherein the skin area comprises at least 50% of the skin surface overlying the melanoma tumor.

Embodiment 13: An embodiment of any of the embodiments of embodiment 1-12, wherein at least 50% of the skin area does not comprise the skin surface overlying the melanoma tumor.

Embodiment 14: An embodiment of any of the embodiments of embodiment 1-13, wherein the adhesive patch is a first adhesive patch, wherein the first adhesive patch is applied to a first skin area, wherein the first skin area comprises a first portion of the skin surface overlying the melanoma tumor, wherein a first portion of the border of the melanoma tumor is determined by comparing the expression levels in each of the plurality of regions of the sample of the first skin area, and wherein the method further comprises: applying a second adhesive patch to a second skin area of the subject, wherein the second skin area comprises a second portion of the skin surface overlying the melanoma tumor, and wherein a portion of the second skin area does not comprise the skin surface overlying the melanoma tumor; removing the adhesive patch from the subject: detecting an expression level of one or both of S100A8 and S100A9 in each of a plurality of regions of a sample of the second skin area adhered to the second adhesive patch; and locating a second portion of the border of the melanoma tumor by comparing the expression levels in each of the plurality of regions of the sample of the second skin area.

Embodiment 15: A method for the surgical removal of a melanoma tumor from a subject, the method comprising: applying an adhesive patch to a skin area of the subject, wherein the skin area comprises at least a portion of a skin surface overlying the melanoma tumor, and wherein a portion of the skin area does not comprise the skin surface overlying the melanoma tumor: removing the adhesive patch from the subject: detecting an expression level of one or both of S100A8 and S100A9 in each of a plurality of regions of a sample of the skin area adhered to the adhesive patch; locating at least a portion of the border of the melanoma tumor by comparing the expression levels in each of the plurality of sample regions; determining, based on the located portion of the border of the melanoma tumor, an outline of a tissue mass to be excised from the subject, wherein the tissue mass comprises at least a portion of the melanoma tumor; and excising the tissue mass from the subject.

Embodiment 16: An embodiment of embodiment 15, wherein the detecting of the expression level comprises measuring a concentration of one or both of an S100A8 RNA sequence and an S100A9 RNA sequence.

Embodiment 17: An embodiment of embodiment 16, wherein the measuring comprises probing the sample region with polynucleotides each independently linked to an imaging agent and having a sequence substantially complementary to that of the S100A8 RNA sequence or the S100A9 RNA sequence.

Embodiment 18: An embodiment of embodiment 15, wherein the detecting of the expression level comprises measuring a concentration of one or both of S100A8 protein and S100A9 protein.

Embodiment 19: An embodiment of embodiment 18, wherein the detecting of the expression level comprises probing the sample region with antibodies each independently linked to an imaging agent and having binding affinity to the S100A8 protein or the S100A9 protein.

Embodiment 20: An embodiment of any of the embodiments of embodiment 15-19, wherein the locating comprises: identifying regions of the skin area, each independently corresponding to one of the plurality of regions of the sample.

Embodiment 21: An embodiment of embodiment 20, wherein the adhesive patch comprises registration marks, wherein the applying of the adhesive patch comprises transferring indicia of registration marks from the adhesive patch to the skin area, and wherein the identifying comprises: correlating the transferred indicia to the registration marks.

Embodiment 22: An embodiment of embodiment 20 or 21, wherein the identifying comprises: classifying a first region of the skin area as a tumor overlying region, wherein the detected expression level in the region of the sample corresponding to the first region of the skin area is greater than a first predetermined threshold; and classifying a second region of the skin area as a surrounding region, wherein the detected expression level in the region of the sample corresponding to the second region of the skin area is less than a second predetermined threshold.

Embodiment 23: An embodiment of embodiment 20 or 21, wherein the identifying comprises: classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold amount.

Embodiment 24: An embodiment of any of the embodiments of embodiment 20 or 21, wherein the identifying comprises: classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold percentage.

Embodiment 25: An embodiment of any of the embodiments of embodiment 22-24, wherein the first region of the skin area and the second region of the skin area share a common edge, and wherein the at least a portion of the border of the melanoma tumor comprises the common edge.

Embodiment 26: An embodiment of any of the embodiments of embodiment 15-25, wherein the skin area comprises at least 50% of the skin surface overlying the melanoma tumor.

Embodiment 27: An embodiment of any of the embodiments of embodiment 15-26, wherein at least 50% of the skin area does not comprise the skin surface overlying the melanoma tumor.

Embodiment 28: A method for the treatment of a melanoma tumor in a subject, the method comprising: applying an adhesive patch to a skin area of the subject, wherein the skin area comprises at least a portion of a skin surface overlying the melanoma tumor, and wherein a portion of the skin area does not comprise the skin surface overlying the melanoma tumor; removing the adhesive patch from the subject; detecting an expression level of one or both of S100A8 and S100A9 in each of a plurality of regions of a sample of the skin area adhered to the adhesive patch: determining, based on the expression level in at least one of the plurality of regions, a dosage amount of a therapeutic agent, wherein the dosage amount is effective for the treatment of the melanoma tumor; and administering to the subject the dosage amount of the therapeutic agent.

Embodiment 29: An embodiment of embodiment 28, wherein the detecting of the expression level comprises measuring a concentration of one or both of an S100A8 RNA sequence and an S100A9 RNA sequence.

Embodiment 30: An embodiment of embodiment 29, wherein the measuring comprises probing the sample region with polynucleotides each independently linked to an imaging agent and having a sequence substantially complementary to that of the S100A8 RNA sequence or the S100A9 RNA sequence.

Embodiment 31: An embodiment of embodiment 28, wherein the detecting of the expression level comprises measuring a concentration of one or both of S100A8 protein and S100A9 protein.

Embodiment 32: An embodiment of embodiment 31, wherein the detecting of the expression level comprises probing the sample region with antibodies each independently linked to an imaging agent and having binding affinity to the S100A8 protein or the S100A9 protein.

Embodiment 33: A method for the treatment of a suspected melanoma tumor in a subject, the method comprising: applying an adhesive patch to a skin area of the subject, wherein the skin area comprises at least a portion of a skin surface overlying the suspected melanoma tumor, wherein the suspected melanoma tumor is not grossly visible, and wherein a portion of the skin area does not comprise the skin surface overlying the suspected melanoma tumor, removing the adhesive patch from the subject; detecting an expression level of one or both of S100A8 and S100A9 in each of a plurality of regions of a sample of the skin area adhered to the adhesive patch; determining, based on the expression level in at least one of the plurality of regions, a dosage amount of a therapeutic agent, wherein the dosage amount is effective for the treatment of the suspected melanoma tumor; and administering to the subject the dosage amount of the therapeutic agent.

Embodiment 34: An embodiment of embodiment 33, wherein the detecting of the expression level comprises measuring a concentration of one or both of an S100A8 RNA sequence and an S100A9 RNA sequence.

Embodiment 35: An embodiment of embodiment 34, wherein the measuring comprises probing the sample region with polynucleotides each independently linked to an imaging agent and having a sequence substantially complementary to that of the S100A8 RNA sequence or the S100A9 RNA sequence.

Embodiment 36: An embodiment of embodiment 33, wherein the detecting of the expression level comprises measuring a concentration of one or both of S100A8 protein and S100A9 protein.

Embodiment 37: An embodiment of embodiment 36, wherein the detecting of the expression level comprises probing the sample region with antibodies each independently linked to an imaging agent and having binding affinity to the S100A8 protein or the S100A9 protein.

### EXAMPLES

The present disclosure will be better understood in view of the following non-limiting examples. The following examples are intended for illustrative purposes only and do not limit in any way the scope of the present invention.

### Example 1. Influence of Cell Type and Case Type on Gene Expression Profile within a Region of Interest

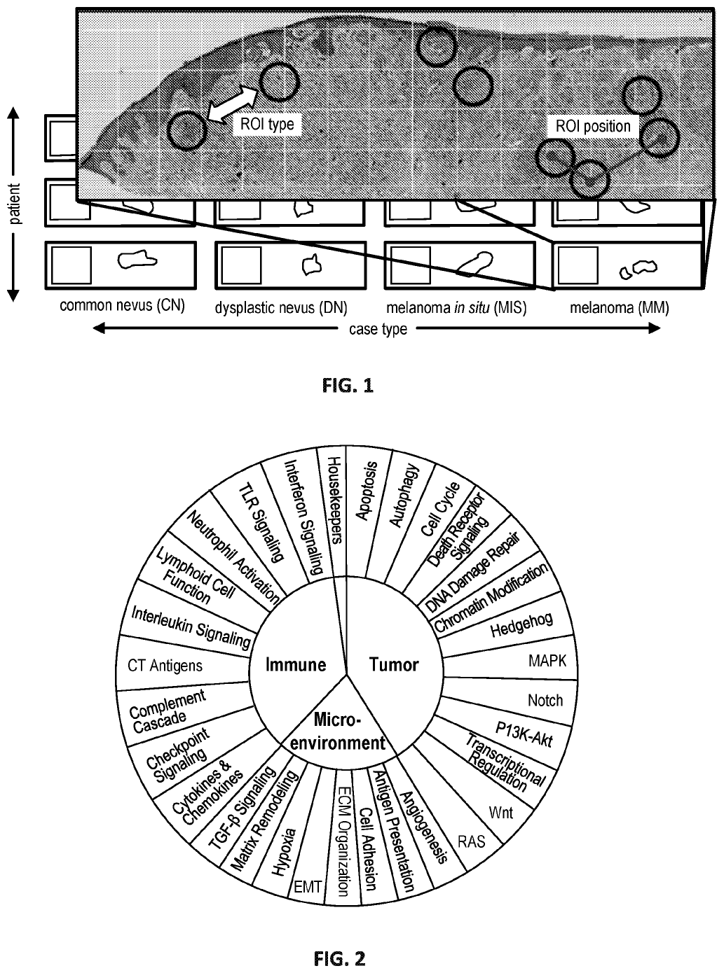

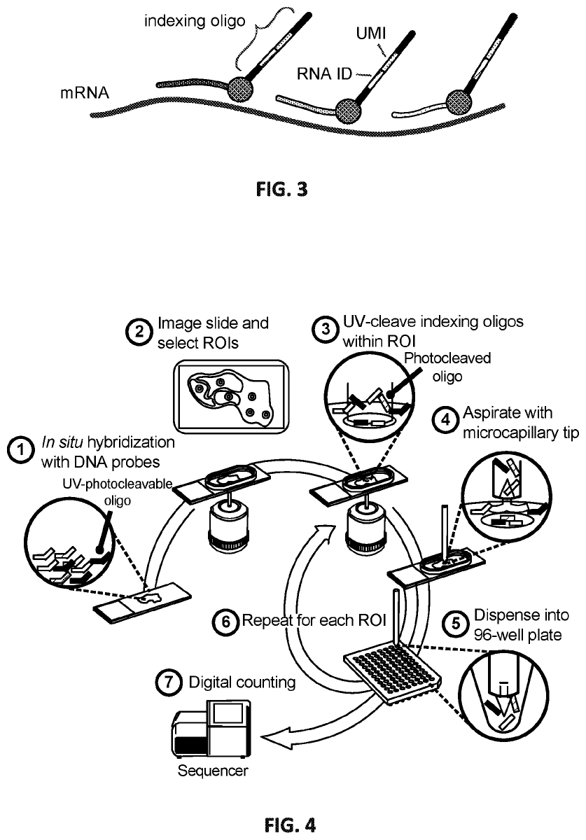

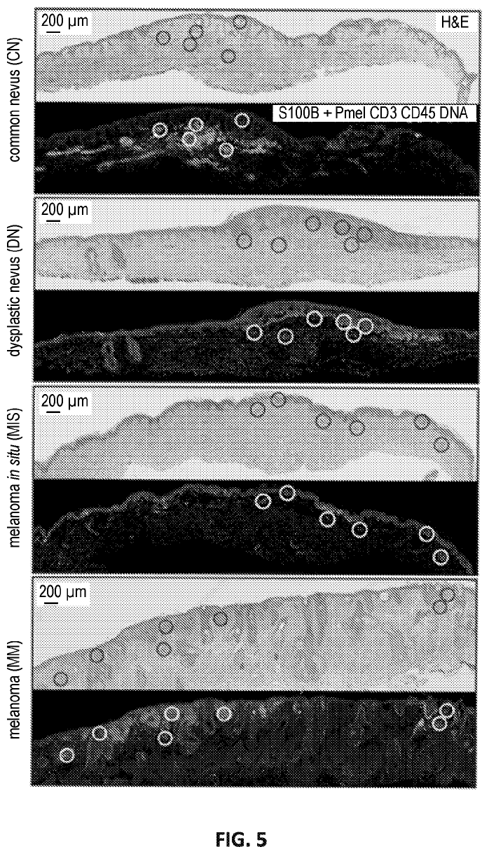

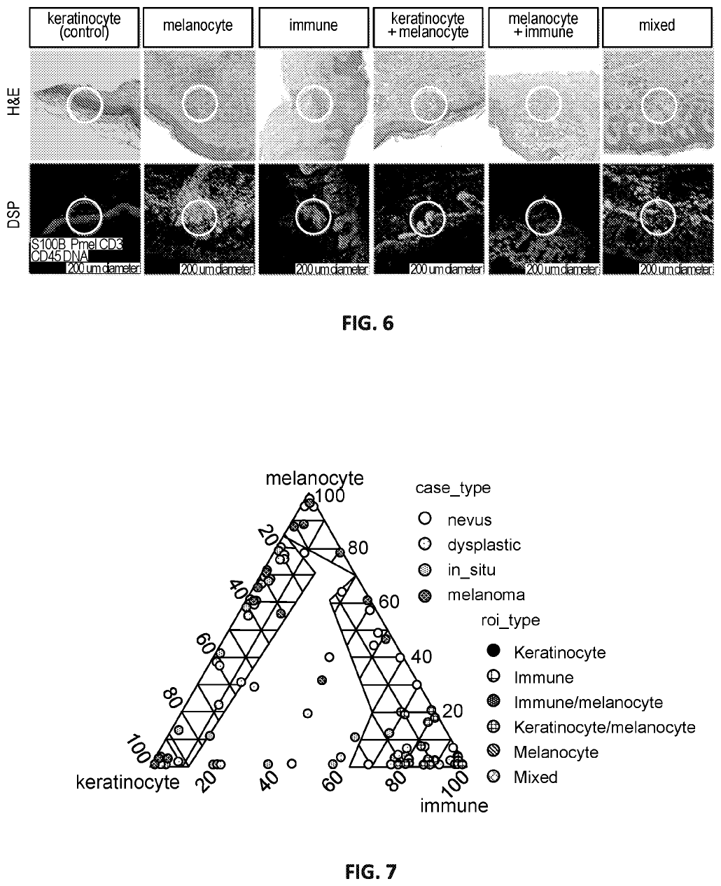

A NanoString GEOMX® Digital Spatial Profiler was used with a 1,412-gene panel (4,998 probes; 3 probes per gene for most genes) to profile three tumors in formalin-fixed paraffin-embedded tissue sections for each of four case types (common nevi, CN; dysplastic nevi, DN; melanoma in situ, MIS; invasive melanoma, MM) as shown in FIGS. 1-5. Two of the three melanoma cases were Stage pT1a and the third was Stage pT2b. Six to sixteen circular regions of interest (ROI), each having a 200-μm diameter, were selected in each tissue, resulting in 134 ROIs in total. One keratinocyte-rich ROI on the periphery of each tissue was chosen to serve as a “normal” control (FIG. 6). To facilitate comparison across tissues, all other ROIs were assigned to one of five categories (immune-rich, melanocyte-rich, immune/melanocyte-rich, keratinocyte/melanocyte-rich or mixed) (FIG. 6) and the cell type composition of each ROI were evaluated (FIG. 7). Raw counts were normalized to the upper quartile (J. H. Bullard, E. Purdham. K. D. Hansen, & S. Dudoit, BMC Bioinformatics 11, (2010): 94) for each ROI to enable comparison across ROIs.

In analyzing the tissue samples, 5-μm-thick serial sections derived from formalin-fixed paraffin-embedded (FFPE) tissue blocks were cut. One section was stained with hematoxylin and eosin (H&E) and two unstained sections were mounted on positively charged histology slides for in situ hybridization and digital spatial profiling (DSP). Whole-slide imaging of the H&E sections was performed on a Nikon TE2000-E microscope and ROIs representative of the tumor and its microenvironment were selected by the pathologist.

For DSP analysis, slides were blocked for 30 min as described (Merritt et al., 2019) and then incubated with 300 nM Sytol3 and fluorescently conjugated antibodies to CD3. S100B, Pmel and CD45 for 1 h and washed in 2×SSC. Slides were loaded onto a DSP instrument and submerged/washed in PBS with 0.1% Tween 20 as described (C. R. Merritt et al., Nat. Biotechnol. 38, (2020): 586). Following 20× fluorescence scanning to obtain a high-resolution image of the tissue, ROIs were selected by matching to the pathologist-selected regions on the H&E-stained serial section. Between six and 16 ROIs were chosen per tissue (all 200-μm diameter circles). Indexing oligos were released from each ROI by exposure to UV light and 10 μl of liquid from above the ROI was collected by a microcapillary tip and deposited in a 96-well plate.

Indexing oligos from each ROI were PCR amplified using primers that 1) hybridize to constant regions and 2) contain unique dual-indexed barcoding sequences to preserve ROT identity. PCR products were pooled and purified twice with Ampure XP beads (Beckman Coulter). Library concentration and purity were measured using a high sensitivity DNA Bioanalyzer chip (Agilent). Paired end (2×75 bp reads) sequencing was performed on an Illumina MiSeq instrument (pilot experiment) or Illumina HiSeq 2500 instrument (full panel experiment). For data analysis, a high confidence detection threshold was set at the geometric mean plus 2.5 standard deviations of the negative probes. 923 of 1,412 genes (65%) in the full panel were above the detection threshold in at least one ROI. The 489 genes below the detection threshold in all ROIs were excluded from further analysis.

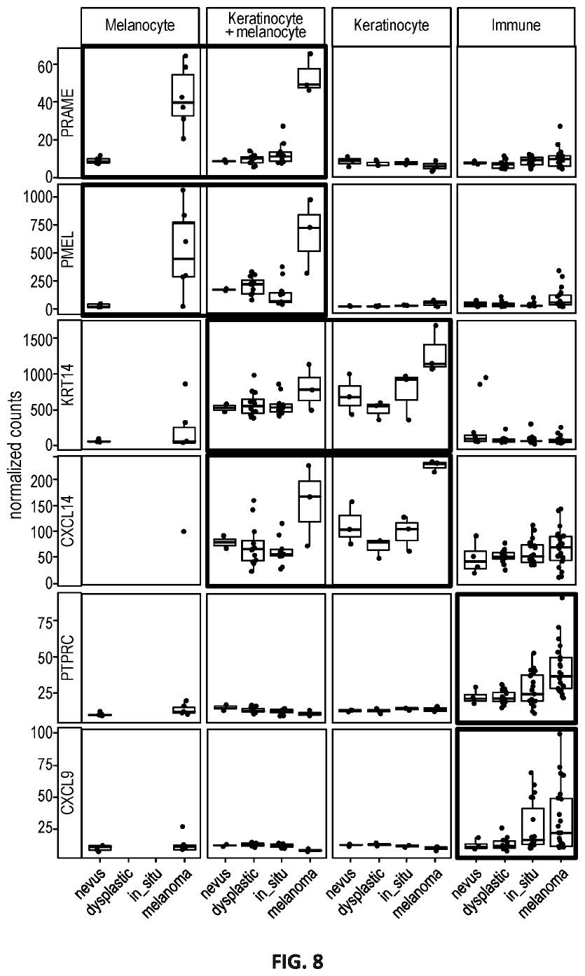

To further validate the performance of DSP, expression of known melanomagenesis-associated genes across the cohort were examined. The melanoma biomarker PRAME (preferentially expressed antigen in melanoma)(L. E. Clarke et al., J. Cutan. Pathol. 42, (2015): 244; P. Gerami et al., J. Am. Acad. Dermatol. 76, (2017): 114) and the melanoma-associated developmental marker PMEL (T. Sarantou et al., Cancer Res. 57, (1997): 1371) was observed to be significantly and specifically elevated in melanocyte-containing ROIs in the three invasive melanoma cases (FIG. 8). KRT/4 and CXCL14 (A. I. Riker et al., BMC Med. Genomics 1, (2008): 13) were elevated in keratinocyte-containing ROIs relative to melanocyte-rich or immune-rich ROIs in all four tumor types and were further upregulated in the melanomas (FIG. 8). Finally, the pan-leukocyte marker PTPRC (CD45) and the T cell chemoattractant gene CXCL9, which are included in a gene expression-based diagnostic test for melanoma (L. E. Clarke et al., J. Cutan. Pathol. 42, (2015)), were detected specifically in immune-rich ROIs, particularly in melanomas (FIG. 8). Together, these results validated prior data and also revealed cell-type specific expression of known melanoma biomarkers.

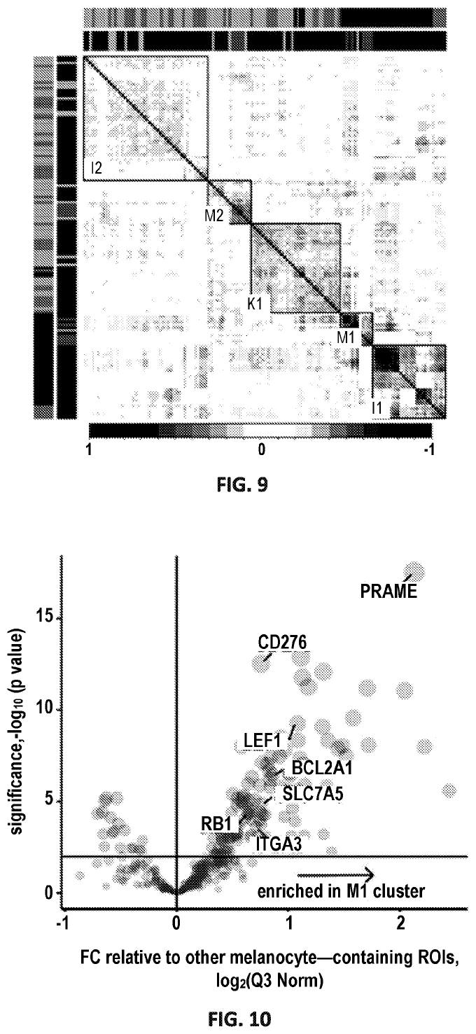

Unbiased clustering of ROIs based on pairwise correlation coefficients revealed that cell type and tumor type both affect the similarity between ROIs (FIG. 9). The ROIs clustered into five groups, two of which consisted of melanocyte-rich ROIs (FIG. 9; M1, M2), two of which consisted immune-rich ROIs (FIG. 9; 11, 12) and one of which consisted of keratinocyte-rich ROIs (FIG. 9; K). For the M and I clusters, the differences between groups 1 and 2 were based on case type: M1 and I1 contained only melanoma or melanoma in situ ROIs, while M2 and 12 had no melanoma ROIs (FIG. 9). The K cluster contained all 12 control keratinocyte ROIs and most of the mixed keratinocyte/melanocyte ROIs, suggesting that keratinocyte-specific genes drive clustering of these ROIs.

### Example 2. Framework for Identification of Cell Type-Specific Gene Expression

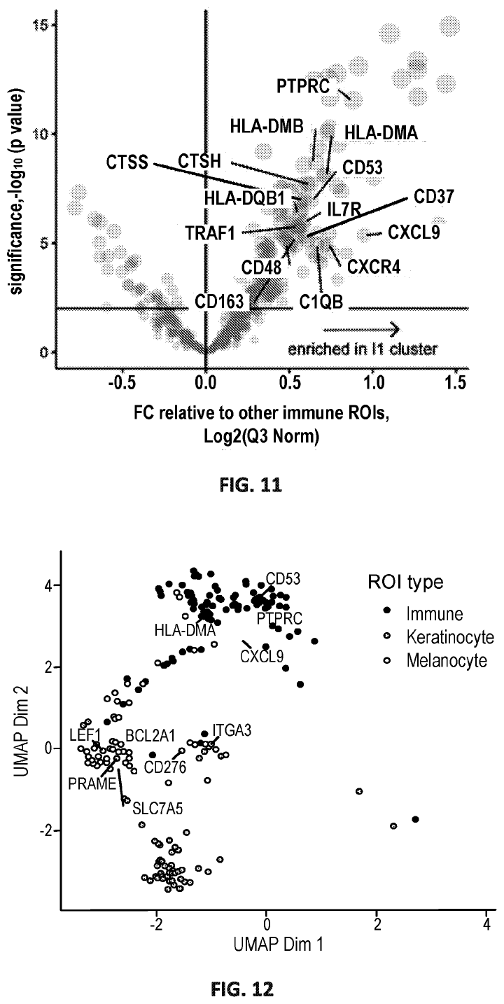

Because the I1 and M1 clusters were composed entirely of ROIs from the melanoma or melanoma in situ cases, genes expressed only in these clusters have biomarker potential. Importantly, since ROIs were selected based on enrichment of certain cell populations, a cell type-specific analysis was possible. Linear regression was performed to identify genes that were 1) significantly enriched in M1 or 11 ROIs compared to other ROIs containing the same cell type(s) and 2) not detected in any ROIs from common nevi. For M1, the known biomarker PRAME was by far the most significantly enriched gene (FIG. 10). The other top hits LEF1, CD276, BCL2A1 and SLC7A5 have been shown to be up-regulated or amplified in melanoma but are also detectable in benign nevi or normal melanocytes (R. Haq et al., Proc. Natl. Acad. Sci. USA 110, (2013): 4321; C. Tekle et al., Int. J. Cancer 130, (2012): 2282: Q. Wang et al., Int. J. Cancer 135, (2014): 1060; S. Xu et al., DNA Cell Biol. 34. (2015): 69). For the immune ROIs in 11, the two most highly enriched genes in melanoma that were also not detected in nevi were PTPRC and CXCL9 (FIG. 11), followed by genes broadly related to leukocyte biology (FIG. 11), including MHC II antigen presentation (HLA-Tx, C7S).

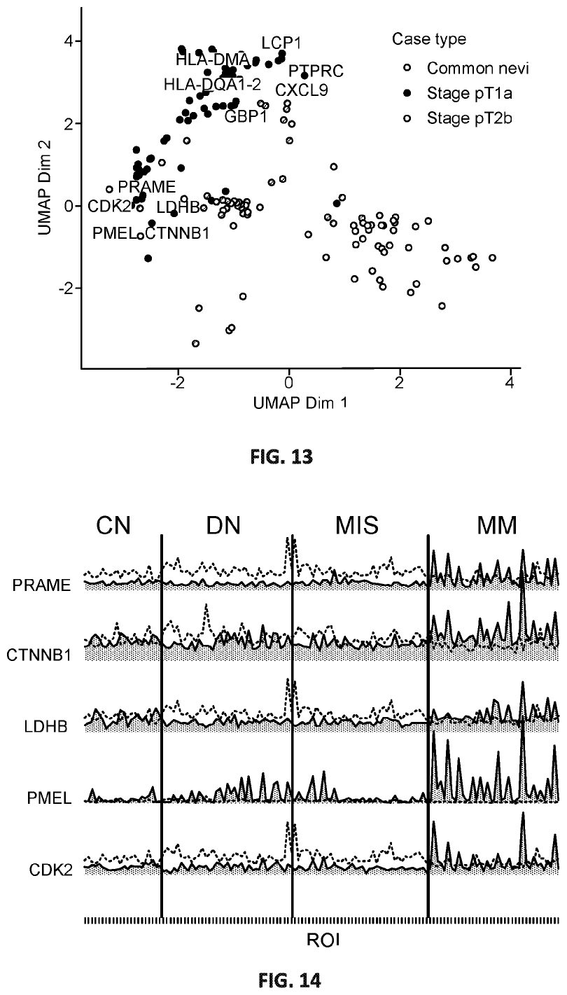

Enrichment analysis does not provide information on the relationship of genes to each other, so to further leverage the spatial resolution of DSP data the dimensionality reduction technique UMAP was used to visualize the relative spatial expression profiles of all genes (FIGS. 12 and 13). This approach can be used to determine which genes tend to be expressed in the same location (or not), a property that is lost in bulk measurements such as RNA-seq. Importantly, marker genes with different spatial expression profiles can provide orthogonal information, whereas genes with the same expression profile are more likely to be reporting on similar biology. The UMAP method was chosen over other methods such as t-SNE because it preserves both local and global data structure and captures meaningful biological relationships (E. Becht et al., Nat. Biotechnol. 37, (2019): 38). At the global level, UMAP analysis produced three gene clusters on the left side of the plot space and the UMAP output was validated by confirming that gene clustering correlated with cell type (FIG. 12, highlighted dots) and tumor type (FIG. 13, highlighted dots).

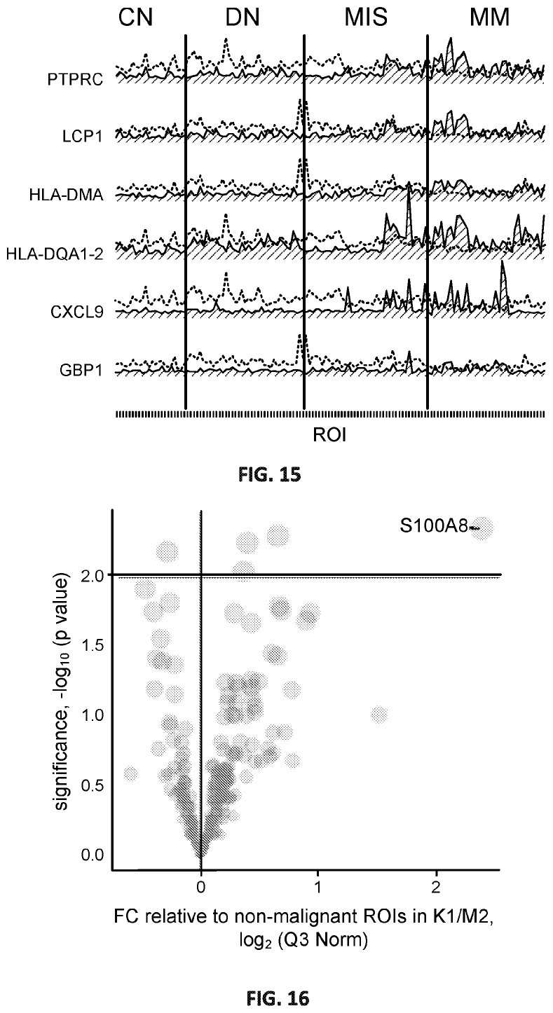

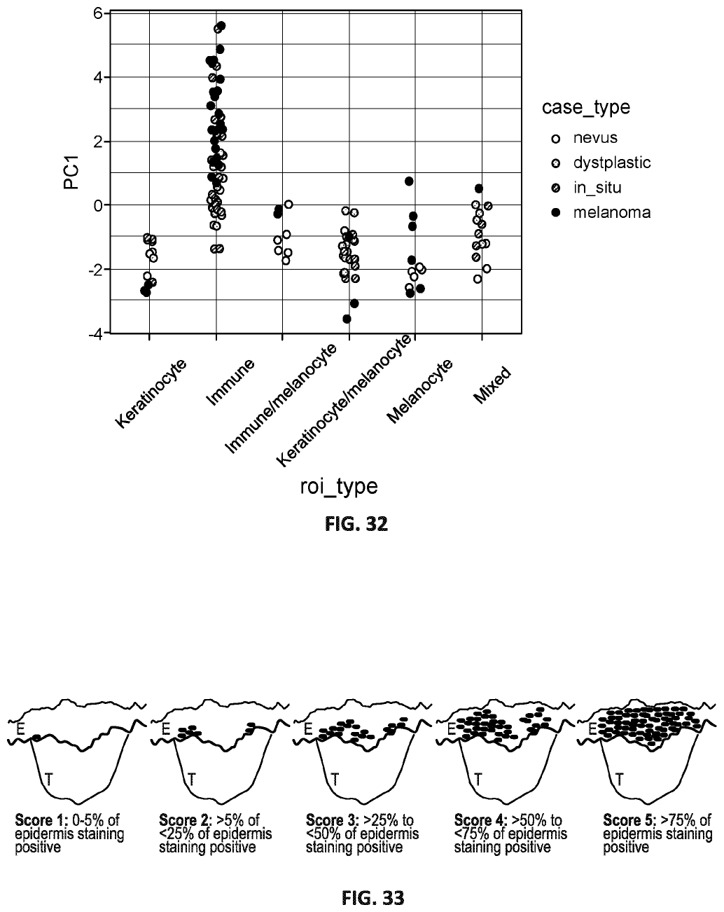

Notably, the expression profiles of PMEL, CTWNNB1, LDHB and CDK2 were more similar to PRAME than the genes enriched in melanoma melanocytes (FIG. 12 vs. FIG. 13), but of these genes only CDK2 was not detected in nevi (FIG. 14). Genes enriched in melanoma immune ROIs were not tightly UMAP-adjacent (FIG. 12); instead they clustered next to genes expressed by the same cell type or within same pathway (FIG. 13; for example, PTPRC and LCP1 are expressed by lymphocytes, HLA-DMA and HLA-DQA1/2 by antigen-presenting cells, CXCL9 with other interferon gamma-stimulated genes such as GBP1). All of these genes were primarily detected in MIS or MM (FIG. 15). The gene signatures shown in FIGS. 14 and 15 were both predictive of cell type and case type (FIGS. 31 and 32). Together, these results established a framework for identification of cell type-specific gene expression during melanoma evolution.

### Example 3. Analysis of Intermediate ROI Clusters Revealing Components of the Keratinocyte Microenvironment in Melanoma

Since the K1/M2 clusters encompass most epidermal keratinocyte-containing ROIs (FIG. 9), it was reasoned that K1/M2 might reveal markers of melanoma in situ, which grows within the epidermis. Linear regression comparing the seven malignant ROIs was performed in vs. all other ROIs in K1 and M2. The most highly enriched gene in this analysis that was also not detected in common nevi was the S100 calcium binding protein family member and DAMP S100A8 (FIG. 16).

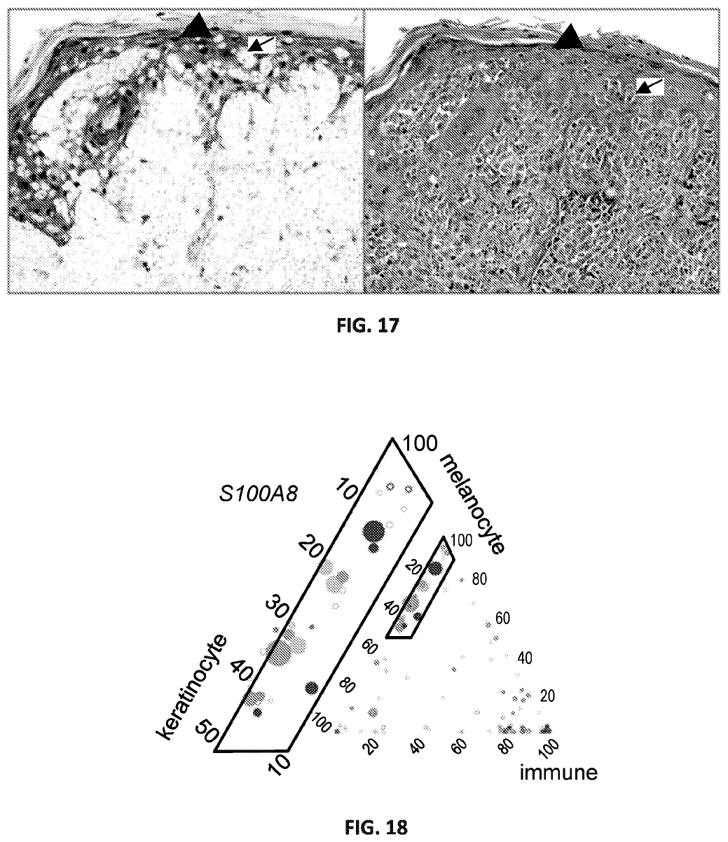

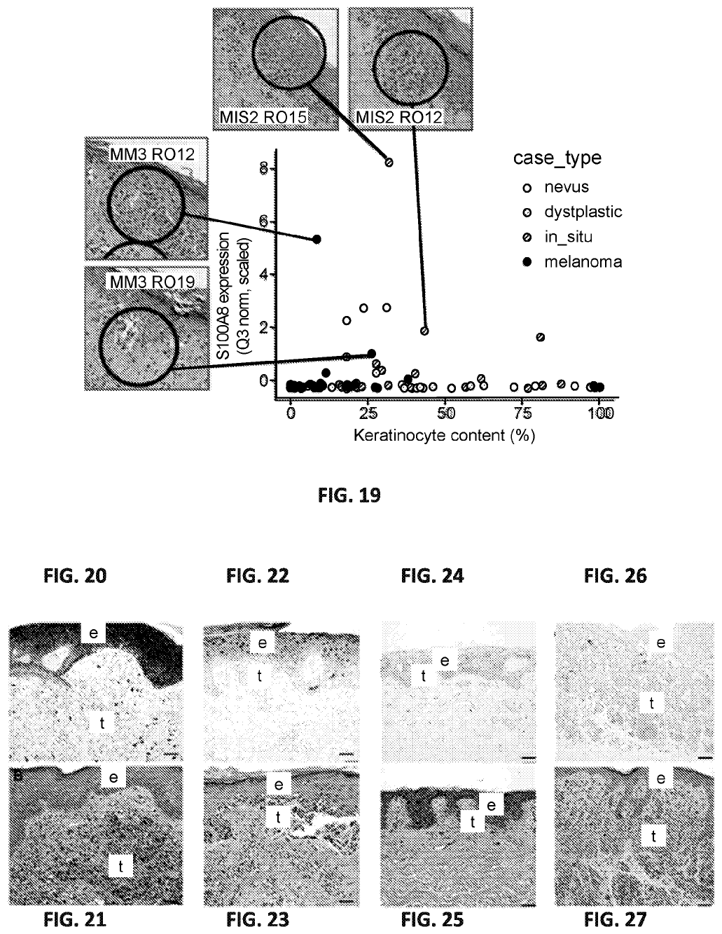

Immunohistochemistry (IHC) was used to determine that S100A8 is expressed by keratinocytes rather than melanocytes (FIG. 17), yet S100A8 was most strongly expressed in ROIs containing >50% melanocytes rather than >50% keratinocytes (FIG. 18), suggesting that keratinocytes may express S100A8 in response to consumption of the epidermis by malignant melanocytes. Indeed, it was observed that the ROIs with the highest S100A8 expression had melanocytes scattered throughout the epidermis, a histopathologic feature of melanoma in situ (FIG. 19). Expression of S00A8 was not strongly correlated with keratinocyte content (FIG. 19), further indicating that high S100A8 expression is not merely a function of keratinocyte abundance. KRT17 and KRT6A-B-C were the closest points to S100A8 in UMAP space, and indeed these genes were enriched in the same ROIs. This profile was not seen for other keratins such as KRT14. KRT17 and KRT6 are known to be upregulated in wounded skin (X. Zhang, M. Yin, & L. J. Zhang. Cells 8, (2019): 807); thus enrichment of these genes in the same ROIs as S100A8 supports the notion that the growth of melanoma in situ within the epidermis elicits an injury response within the keratinocytes.

For the IHC analyses, S100A8 mouse monoclonal antibody (catalog #14-9745-82. eBioscience) and S100A9 mouse monoclonal antibody (catalog #MAB5578-SP, R&D Systems) were used. Staining was scored with 100% consensus agreement by two observers based on the percentage of epidermis stained that is directly associated with the tumor: score 1 =0-5%, score 2=6% to 25%; score 3=26% to 50%; score 4=51% to 75%; score 5=>75%.

### Example 4. Detection of S100A8/A9 in the Keratinocyte Microenvironment of Melanoma

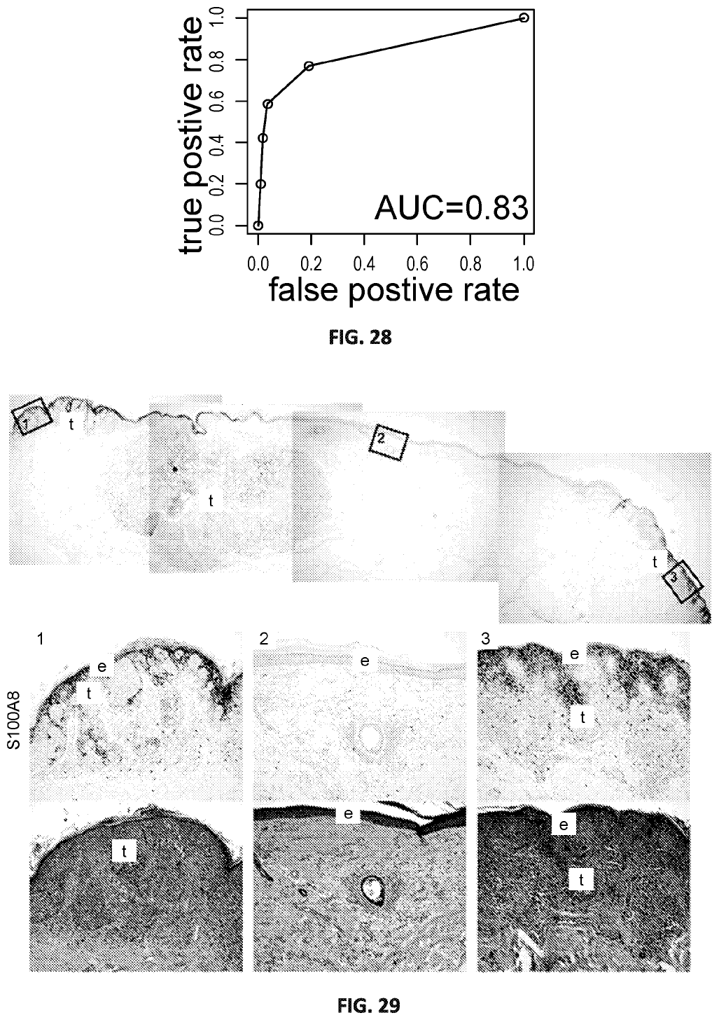

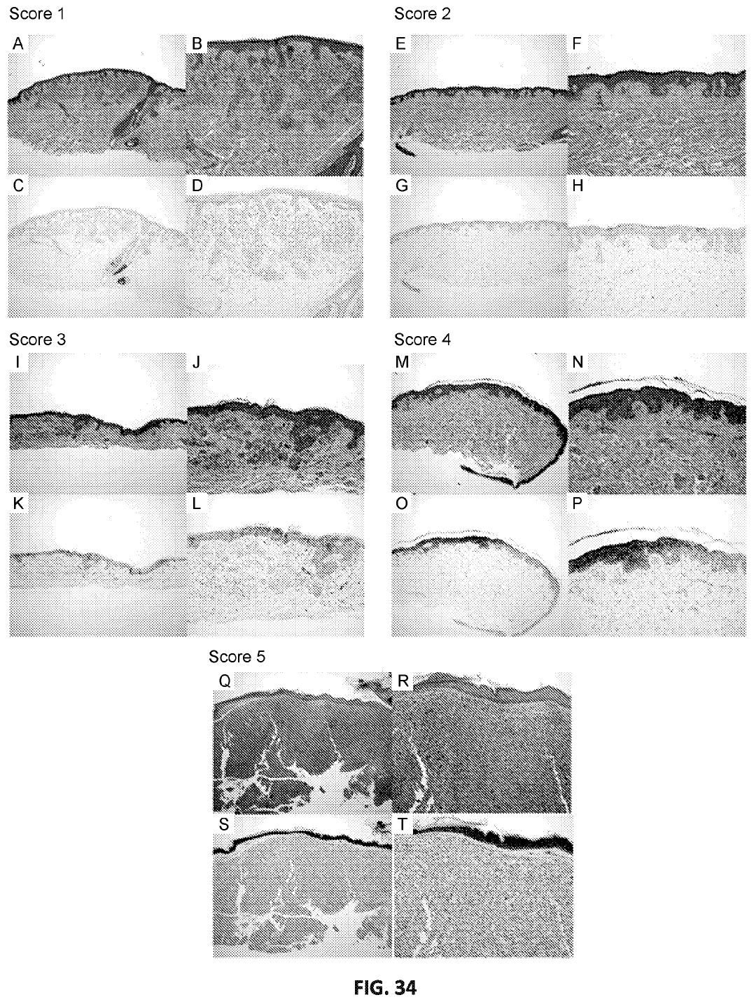

To further validate the data from spatial transcript profiling, S100A8 IHC was performed on an independent cohort of 252 melanocytic tumors (68 CN, 66 DN, 69 MIS. 49 MM; Table 1). S100A8 expression was scored as shown in FIG. 33 based on the percentage of epidermis expressing S100A8 that was directly associated with the tumor (epidermis containing tumor and/or epidermis overlying intradermal tumor). Notably, the keratinocyte microenvironment of melanoma (FIGS. 20 and 21) and many cases of melanoma in situ (FIGS. 22 and 23) showed prominent expression of S100A8, while the keratinocyte microenvironment of most dysplastic nevi (FIGS. 24 and 25) and common nevi (FIGS. 26 and 27) lacked or had only limited staining (Table 2, p<0.001; FIG. 28, AUC 0.83; representative images of scores 1-5 are presented in FIG. 34). A binary logistic regression model showed that increased S100A8 IHC score was significantly associated with invasive melanoma tumor type (OR=2.49, 95% CI 1.93-3.21) and remained significant after adjusting for sex, anatomic site and age (OR=2.54, 95% CI 1.92-3.36). S100A8 expression was also apparent in scattered immune cells within the dermis of both nevi and melanoma as well as within the normal hair follicle epithelium, but this expression was not taken into account when scoring S100A8 staining. Remarkably, if the tumor showed “skip areas,” i.e., areas of uninvolved skin, the epidermis lacked S100A8 expression in these foci (FIG. 29).

TABLE 1
Patient and tumor characteristics of a cohort of 252 tumors
stained for S100A8 by immunohistochemistry.

| | Common | Dysplastic | Melanoma | Invasive | |
|---|---|---|---|---|---|
| | nevus | nevus | in situ | melanoma | Total |
| Total | 68 | 66 | 69 | 49 | 252 |
| Sex | | | | | |
| Male | 28 (41.2%) | 35 (53.0%) | 39 (56.5%) | 33 (67.3%) | 135 (53.6%) |
| Female | 40 (58.8%) | 31 (47.0%) | 30 (43.5%) | 16 (32.7%) | 117 (46.4%) |
| Average age (years) | 44.1 | 52.8 | 62.5 | 62.2 | 55.0 |
| Location of tumor | | | | | |
| Face | 5 (7.4%) | 1 (1.5%) | 10 (14.5%) | 7 (14.3%) | 23 (9.1%) |
| Scalp or neck | 9 (13.2%) | 0 | 5 (7.2%) | 5 (10.2%) | 19 (7.5%) |
| Trunk | 39 (57.4%) | 49 (74.2%) | 22 (31.9%) | 12 (24.5%) | 122 (48.3%) |
| Upper extremity | 5 (7.4%) | 11 (16.7%) | 24 (34.8%) | 13 (26.5%) | 53 (21.0%) |
| Lower extremity | 10 (14.7%) | 5 (7.6%) | 7 (10.1%) | 12 (24.5%) | 34 (13.5%) |
| Unknown | 0 | 0 | 1 (1.4%) | 0 | 1 (0.4%) |
| Nevis growth pattern | | | | | |
| Junctional | 7 (10.3%) | 38 (57.6%) | n/a | n/a | n/a |
| Compound | 30 (44.1%) | 28 (42.4%) | n/a | n/a | n/a |
| Intradermal | 31 (45.650 | 0 | n/a | n/a | n/a |
| Invasive melanoma | n/a | n/a | n/a | 2.33 | n/a |
| average thickness (mm) | | | | | |
| Invasive melanoma | n/a | n/a | n/a | 5 (10.2%) | n/a |
| ulceration present | | | | | |
| Primary tumor stage | | | | | |
| Tis | n/a | n/a | 69 | n/a | n/a |
| T1a | n/a | n/a | n/a | 29 (5.2%) | n/a |
| T1b | n/a | n/a | n/a | 5 (10.2%) | n/a |
| T2a | n/a | n/a | n/a | 2 (4.1%) | n/a |
| T2b | n/a | n/a | n/a | 2 (4.1%) | n/a |
| T3a | n/a | n/a | n/a | 5 (10.2%) | n/a |
| T3b | n/a | n/a | n/a | 1 (2.0%) | n/a |
| T4a | n/a | n/a | n/a | 3 (6.1%) | n/a |
| T4b | n/a | n/a | n/a | 2 (4.1%) | n/a |

TABLE 2
S100A8 expression in a cohort of 252 tumors.

| | Common | Dysplastic | Melanoma | Invasive | |
|---|---|---|---|---|---|
| | nevus | nevus | in situ | melanoma | Total |
| Total | 68 | 66 | 69 | 49 | 252 |
| S100A8 IHC score * | | | | | |
| Score 1 (0-4%) | 51 (75.0%) | 48 (72.7%) | 21 (30.4%) | 3 (6.1%) | 123 (48.8%) |
| Score 2 (5-25%) | 15 (22.1%) | 13 (19.7%) | 13 (18.8%) | 7 (14.3%) | 48 (19.0%) |
| Score 3 (26-50%) | 1 (1.5%) | 2 (3.0%) | 8 (11.6%) | 10 (20.4%) | 21 (8.3%) |
| Score 4 (51-75%) | 0 | 2 (3.0%) | 19 (27.5%) | 12 (24.5%) | 33 (13.1%) |
| Score 5 (>75%) | 1 (1.5%) | 1 (1.5%) | 8 (11.6%) | 17 (34.7%) | 27 (10.7%) |

* Percent of epidermis associated with tumor stained; Pearson Chi-Sqaure P-value of 0.001

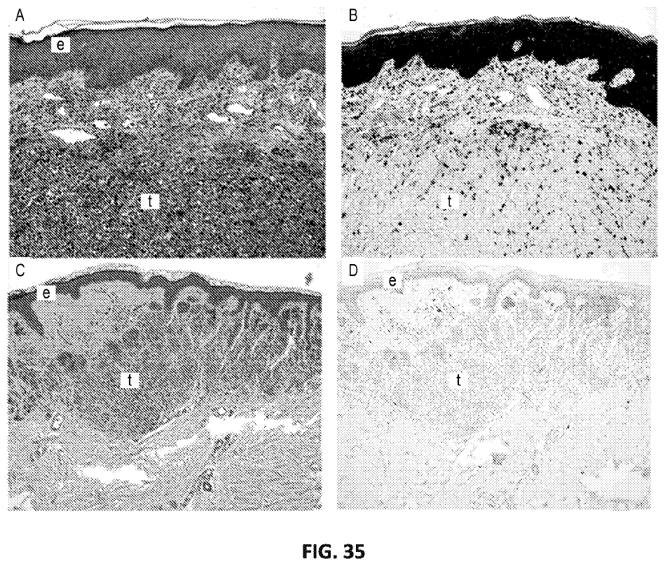

S100A8 is generally co-expressed with S100A9, forming a complex known as calprotectin (C. Gebhardt, J. Nemeth, P. Angel, & J. Hess, Biochem. Pharmacol. 72, (2006): 1622). Because the DSP probe pool used in these experiments did not target S100A9 and the S10A8 antibody (CF-145) used is not cross-reactive, the expression of S100A9 was analyzed in a subset of tumors by immunohistochemistry. Similar to S100A8, S100A9 was expressed by the keratinocytes associated with melanoma but not nevi (FIG. 35, Table 3). Together, these data demonstrate that epidermal keratinocytes express S100A8/A9 in response to melanoma growth, revealing the cellular origin of a melanoma biomarker within the tumor microenvironment and emphasizing the interaction between melanoma and the keratinocyte microenvironment.

TABLE 3
S100A9 staining results.

| S100A9 score* | Common nevi (N) | Melanoma (N) | |
|---|---|---|---|
| Score 1 | 6 | 0 | P = 0.01 |
| Score 2 | 2 | 0 | |
| Score 3 | 1 | 2 | |
| Score 4 | 0 | 3 | |
| Score 5 | 0 | 4 | |

*Percent of epidermis associated with tumor stained

In summary, the Examples described above provide information related to tumor-microenvironment interactions in melanoma within their native morphological context using GeoMx DSP. After validating the performance and reproducibility of the DSP assay on our FFPE tissue sections, 134 ROIs were profiled in a cohort of 12 common benign and malignant melanocytic tumors with a panel that targets 1,400+ immune-oncology-related genes. Over 900 targets were identified with high confidence in a single experiment, which would traditionally require tedious microdissection or be limited to a few genes (F. Wang et al., J. Mol. Diagn. 14, (2012): 22). While other technologies for transcriptome-scale spatial profiling have been applied to melanoma, the experiments described herein detected approximately ten times more transcripts per square micron than in another recent study (K Thrane, H. Eriksson, J. Maaskola, J. Hansson. & J. Lundeberg, Cancer Res. 78, (2018): 5970). Importantly, the compatibility of GeoMx DSP with FFPE enables profiling of archival tissues (all specimens in the study were at least two years old), which has been especially difficult for skin samples (L. N. Kwong et al., JCO Precis. Oncol. 2, (2018): PO.17.00259). This is particularly important for the study of melanoma evolution in patient-derived benign and malignant primary tumors and the keratinocyte microenvironment, which have been mainly overlooked in scRNAseq studies of melanoma. (L. Jerby-Amon et al., Cell 175, (2018): 984; I. Tirosh et al., Science 352, (2016): 189)

A cell type-enriched ROI selection strategy enabled the direct comparison of similar cell populations across tumors when looking for gene enrichment in melanomas. This approach, coupled with a dimensionality reduction-based approach to identify co-expressed genes, successfully identified known melanoma-associated markers and demonstrated their specificity to melanocytes, immune infiltrates or the epidermal (keratinocyte) microenvironment. Interestingly, genes enriched in melanocytes (PRAME), keratinocytes (S100A8) or immune cells (PTPRC. CXCL9) in the study correspond with the three components of a commercially available diagnostic test: the results may explain why the three components do not correlate well with each other and are all required to generate the most predictive score (L. E. Clarke et al., J. Cutan. Pathol. 42, (2015): 244).

Furthermore, a published bulk RNA-seq dataset comparing 57 melanomas and 23 nevi (M. Kunz et al., Oncogene 37, (2018): 6136) both validates the data presented herein and highlights the advantages of a spatially resolved, cell type-specific analysis, especially regarding the tumor microenvironment. While the melanoma markers specific to melanocyte ROIs were also melanoma-enriched in the bulk RNA-seq data, markers of the immune and keratinocyte microenvironment were not as strongly melanoma-associated, likely reflecting masking of less abundant cell types in bulk measurements. By contrast, enrichment of these gene sets in melanoma was of similar magnitude for melanocyte-versus immune-associated genes in the DSP data, suggesting that this technology may be more sensitive for identifying gene products that originate from non-tumor cells in the tumor microenvironment. In fact, a recent study using GeoMx DSP to study protein expression in melanoma found that expression of PD-L1 in macrophages, rather than tumor cells, was predictive of response to immunotherapy (M. I. Toki et al., Clin. Cancer Res. 25. (2019): 5503). Because understanding of tumor-microenvironment interactions in melanoma is much needed in the era of melanoma immunotherapy, additional studies can be used to validate the genes enriched in melanoma immune infiltrates in the DSP cohort.

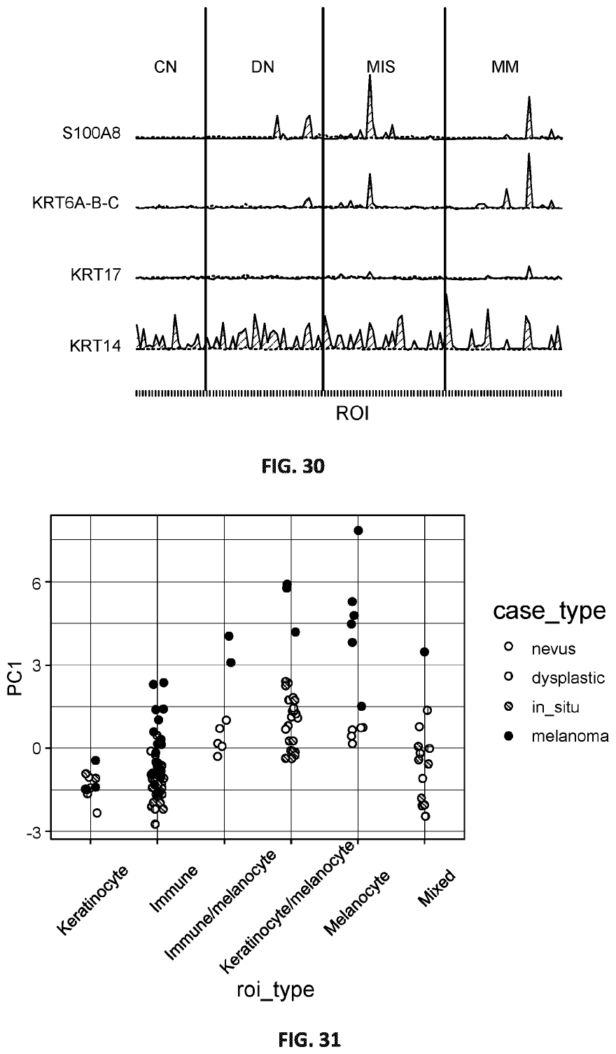

Due to the correlation of S100A8 expression with melanocyte growth within the epidermis (FIGS. 17-19) and expression of the wound-associated keratins 6 and 17 (FIG. 30), S100A8 expression in this context can be a response to inflammation or destruction of the epidermis by the melanocytes. This hypothesis is supported by literature describing S100A8 being expressed in epithelial cells in response to stress and inflammation (C. Kerkhoff et al., Exp. Dermatol. 21, (2012): 822). Cytokines secreted by nearby tumor cells likely play a role as well, since keratinocytes overlying melanoma purely within the dermis also strongly expressed S100A8 and multiple cytokines are known to induce S100A8 in normal keratinocytes (T. Nukui et al., J. Cell Biochem. 104, (2008): 453). Because S100A8/A9 is a chemoattractant for melanocytes that express certain cell adhesion molecules such as MCAM, ALCAM and RAGE (I. M. Ruma et al., Clin. Exp. Metastasis 33, (2016): 609), induction of S100A8 in the epidermis can stimulate melanocyte migration; indeed, S100A8/A9 have been implicated in metastasis of melanoma and other tumor types (K. Arai et al., Curr. Cancer Drug Targets 8, (2008): 243; 1. M. Ruma et al., Clin. Exp. Metastasis 33, (2016): 609; A. Saha et al., J. Biol. Chem. 285, (2010): 10822). Additional DSP studies profiling a larger number of patients and ROIs can further resolve the interplay between keratinocytes and melanocytes during melanomagenesis.

The terms “first” and “second” when used herein with reference to sample regions, skin area regions, sample skin areas, skin surface portions, adhesive patches, border portions, and thresholds, are simply to more clearly distinguish the two elements or properties and are not intended to indicate order.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A method of mapping a border of a melanoma tumor in a subject, the method comprising:
applying an adhesive patch to a skin area of the subject, wherein the skin area comprises at least a portion of a skin surface overlying the melanoma tumor, and wherein a portion of the skin area does not comprise the skin surface overlying the melanoma tumor;
removing the adhesive patch from the subject;
detecting an expression level of one or both of S100A8 and S100A9 in each of a plurality of regions of a sample of the skin area adhered to the adhesive patch; and
locating at least a portion of the border of the melanoma tumor by comparing the expression levels in each of the plurality of sample regions.

2. The method of claim 1, wherein the detecting of the expression level comprises measuring a concentration of one or both of an S100A8 RNA sequence and an S100A9 RNA sequence.

3. The method of claim 2, wherein the measuring comprises probing the sample region with polynucleotides each independently linked to an imaging agent and having a sequence substantially complementary to that of the S100A8 RNA sequence or the S100A9 RNA sequence.

4. The method of claim 1, wherein the detecting of the expression level comprises measuring a concentration of one or both of S100A8 protein and S100A9 protein.

5. The method of claim 4, wherein the detecting of the expression level comprises probing the sample region with antibodies each independently linked to an imaging agent and having binding affinity to the S100A8 protein or the S100A9 protein.

6. The method of claim 1, wherein the locating comprises:
identifying regions of the skin area, each independently corresponding to one of the plurality of regions of the sample.

7. The method of claim 6, wherein the adhesive patch comprises registration marks, wherein the applying of the adhesive patch comprises transferring indicia of registration marks from the adhesive patch to the skin area, and wherein the identifying comprises:
correlating the transferred indicia to the registration marks.

8. The method of claim 6, wherein the identifying comprises:
classifying a first region of the skin area as a tumor overlying region, wherein the detected expression level in the region of the sample corresponding to the first region of the skin area is greater than a first predetermined threshold; and
classifying a second region of the skin area as a surrounding region, wherein the detected expression level in the region of the sample corresponding to the second region of the skin area is less than a second predetermined threshold.

9. The method of claim 6, wherein the identifying comprises:
classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold amount.

10. The method of claim 6, wherein the identifying comprises:
classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold percentage.

11. The method of claim 8, wherein the first region of the skin area and the second region of the skin area share a common edge, and wherein the at least a portion of the border of the melanoma tumor comprises the common edge.

12. The method of claim 1, wherein the skin area comprises at least 50% of the skin surface overlying the melanoma tumor.

13. The method of claim 1, wherein at least 50% of the skin area does not comprise the skin surface overlying the melanoma tumor.

14. The method of claim 1, wherein the adhesive patch is a first adhesive patch, wherein the first adhesive patch is applied to a first skin area, wherein the first skin area comprises a first portion of the skin surface overlying the melanoma tumor, wherein a first portion of the border of the melanoma tumor is determined by comparing the expression levels in each of the plurality of regions of the sample of the first skin area, and wherein the method further comprises:
applying a second adhesive patch to a second skin area of the subject, wherein the second skin area comprises a second portion of the skin surface overlying the melanoma tumor, and wherein a portion of the second skin area does not comprise the skin surface overlying the melanoma tumor;
removing the adhesive patch from the subject;
detecting an expression level of one or both of S100A8 and S100A9 in each of a plurality of regions of a sample of the second skin area adhered to the second adhesive patch; and
locating a second portion of the border of the melanoma tumor by comparing the expression levels in each of the plurality of regions of the sample of the second skin area.

15. A method for the surgical removal of a melanoma tumor from a subject, the method comprising:
applying an adhesive patch to a skin area of the subject, wherein the skin area comprises at least a portion of a skin surface overlying the melanoma tumor, and wherein a portion of the skin area does not comprise the skin surface overlying the melanoma tumor;
removing the adhesive patch from the subject;
detecting an expression level of one or both of S100A8 and S100A9 in each of a plurality of regions of a sample of the skin area adhered to the adhesive patch;
locating at least a portion of the border of the melanoma tumor by comparing the expression levels in each of the plurality of sample regions;
determining, based on the located portion of the border of the melanoma tumor, an outline of a tissue mass to be excised from the subject, wherein the tissue mass comprises at least a portion of the melanoma tumor; and
excising the tissue mass from the subject.

16. The method of claim 15, wherein the detecting of the expression level comprises measuring a concentration of one or both of an S100A8 RNA sequence and an S100A9 RNA sequence.

17. The method of claim 16, wherein the measuring comprises probing the sample region with polynucleotides each independently linked to an imaging agent and having a sequence substantially complementary to that of the S100A8 RNA sequence or the S100A9 RNA sequence.

18. The method of claim 15, wherein the detecting of the expression level comprises measuring a concentration of one or both of S100A8 protein and S100A9 protein.

19. The method of claim 18, wherein the detecting of the expression level comprises probing the sample region with antibodies each independently linked to an imaging agent and having binding affinity to the S100A8 protein or the S100A9 protein.

20. The method of claim 15, wherein the locating comprises:
identifying regions of the skin area, each independently corresponding to one of the plurality of regions of the sample.

21. The method of claim 20, wherein the adhesive patch comprises registration marks, wherein the applying of the adhesive patch comprises transferring indicia of registration marks from the adhesive patch to the skin area, and wherein the identifying comprises:
correlating the transferred indicia to the registration marks.

22. The method of claim 20, wherein the identifying comprises:
classifying a first region of the skin area as a tumor overlying region, wherein the detected expression level in the region of the sample corresponding to the first region of the skin area is greater than a first predetermined threshold; and
classifying a second region of the skin area as a surrounding region, wherein the detected expression level in the region of the sample corresponding to the second region of the skin area is less than a second predetermined threshold.

23. The method of claim 20, wherein the identifying comprises:
classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold amount.

24. The method of claim 20, wherein the identifying comprises:
classifying a first region of the skin area as a tumor overlying region and a second region of the skin area as a surrounding region, wherein the detected expression level in the region of the sample corresponding to the first region of the skin area exceeds the detected expression level in the region of the sample corresponding to the second region of the skin area by greater than a predetermined threshold percentage.

25. The method of claim 22, wherein the first region of the skin area and the second region of the skin area share a common edge, and wherein the at least a portion of the border of the melanoma tumor comprises the common edge.

26. The method of claim 15, wherein the skin area comprises at least 50% of the skin surface overlying the melanoma tumor.

27. The method of claim 15, wherein at least 50% of the skin area does not comprise the skin surface overlying the melanoma tumor.

28. A method for the treatment of a melanoma tumor in a subject, the method comprising:
applying an adhesive patch to a skin area of the subject, wherein the skin area comprises at least a portion of a skin surface overlying the melanoma tumor, and wherein a portion of the skin area does not comprise the skin surface overlying the melanoma tumor;
removing the adhesive patch from the subject;
detecting an expression level of one or both of S100A8 and S100A9 in each of a plurality of regions of a sample of the skin area adhered to the adhesive patch;
determining, based on the expression level in at least one of the plurality of regions, a dosage amount of a therapeutic agent, wherein the dosage amount is effective for the treatment of the melanoma tumor; and
administering to the subject the dosage amount of the therapeutic agent.

29. The method of claim 28, wherein the detecting of the expression level comprises measuring a concentration of one or both of an S100A8 RNA sequence and an S100A9 RNA sequence.

30. The method of claim 29, wherein the measuring comprises probing the sample region with polynucleotides each independently linked to an imaging agent and having a sequence substantially complementary to that of the S100A8 RNA sequence or the S100A9 RNA sequence.

31. The method of claim 28, wherein the detecting of the expression level comprises measuring a concentration of one or both of S100A8 protein and S100A9 protein.

32. The method of claim 31, wherein the detecting of the expression level comprises probing the sample region with antibodies each independently linked to an imaging agent and having binding affinity to the S100A8 protein or the S100A9 protein.

33. A method for the treatment of a suspected melanoma tumor in a subject, the method comprising:
applying an adhesive patch to a skin area of the subject, wherein the skin area comprises at least a portion of a skin surface overlying the suspected melanoma tumor, wherein the suspected melanoma tumor is not grossly visible, and wherein a portion of the skin area does not comprise the skin surface overlying the suspected melanoma tumor;
removing the adhesive patch from the subject;
detecting an expression level of one or both of S100A8 and S100A9 in each of a plurality of regions of a sample of the skin area adhered to the adhesive patch;
determining, based on the expression level in at least one of the plurality of regions, a dosage amount of a therapeutic agent, wherein the dosage amount is effective for the treatment of the suspected melanoma tumor; and
administering to the subject the dosage amount of the therapeutic agent.

34. The method of claim 33, wherein the detecting of the expression level comprises measuring a concentration of one or both of an S100A8 RNA sequence and an S100A9 RNA sequence.

35. The method of claim 34, wherein the measuring comprises probing the sample region with polynucleotides each independently linked to an imaging agent and having a sequence substantially complementary to that of the S100A8 RNA sequence or the S100A9 RNA sequence.

36. The method of claim 33, wherein the detecting of the expression level comprises measuring a concentration of one or both of S100A8 protein and S100A9 protein.

37. The method of claim 36, wherein the detecting of the expression level comprises probing the sample region with antibodies each independently linked to an imaging agent and having binding affinity to the S100A8 protein or the S100A9 protein.
